(19) United States
(12) Patent Application Publication
(10) Pub. No.: US 2021/0035301 A1
(43) Pub. Date: Feb. 4, 2021

(54) INFORMATION PROCESSING SYSTEM, INFORMATION PROCESSING APPARATUS, RECORDING MEDIUM, AND INFORMATION PROCESSING METHOD

(71) Applicants: NIKON CORPORATION, Tokyo (JP); Optos PLC, Dunfermline Fife (GB)

(72) Inventors: Devin SOARES, Marlborough, MA (US); Branden COLEMAN, Marlborough, MA (US); Bradley YATES, Marlborough, MA (US); Naoyuki KAWACHI, Tokyo (JP)

(73) Assignees: NIKON CORPORATION, Tokyo (JP); Optos plc, Dunfermline Fife (GB)

(21) Appl. No.: 16/943,618

(22) Filed: Jul. 30, 2020

Related U.S. Application Data
(60) Provisional application No. 62/880,950, filed on Jul. 31, 2019.

(51) Int. Cl. G06T 7/00; G06F 3/0482; G06F 3/0484; G16H 30/40; G16H 50/20; G16H 40/67; G16H 10/60

(52) U.S. Cl. CPC G06T 7/0016; G06F 3/0482; G06F 3/04847; G16H 30/40; G06T 2200/24; G16H 40/67; G16H 10/60; G06T 2207/30041; G16H 50/20

(57) ABSTRACT

An information processing apparatus comprises: a processor configured to execute a program; and a storage device configured to store the program, the processor being configured to execute: obtaining processing of obtaining a diagnosis result of both eyes; generation processing of generating diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; and output processing of outputting the diagnosis result display information generated by the generation processing.

### CLAIM OF PRIORITY

The present application claims priority from U.S. provisional application 62/880,950 filed on Jul. 31, 2019, the content of which is hereby incorporated by reference into this application.

### BACKGROUND

The present invention relates to an information processing system, an information processing apparatus, a recoeding medium, and an information processing method.

It is known that at an ophthalmic checkup, diagnosis results of both eyes are displayed (see JP2010-5152A). However, in JP2010-5152 A, diagnosis results such as intraocular pressure and visual acuity are independently displayed for each subject eye, thus lowering the visibility of the diagnosis results.

### SUMMARY

First disclosure of an information processing system is an information processing system comprising: a first information processing apparatus which stores subject eye image data of both eyes of a patient; a second information processing apparatus which is communicably connected with the first information processing apparatus; and a third information processing apparatus which is communicably connected with the second information processing apparatus, wherein the second information processing apparatus is configured to execute first transfer processing of receiving the subject eye image data of the both eyes of the patient from the first information processing apparatus and transmitting the subject eye image data to the third information processing apparatus, the third information processing apparatus is configured to execute diagnosis processing of executing image diagnosis based on the subject eye image data of the both eyes transferred by the first transfer processing, and transmitting a diagnosis result of the both eyes to the second information processing apparatus, the second information processing apparatus is configured to execute: obtaining processing of obtaining the diagnosis result of the both eyes transmitted by the diagnosis processing, and generation processing of generating diagnosis result display information and transmitting the diagnosis result display information to the first information processing apparatus, the diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye, and the first information processing apparatus is configured to execute output processing of outputting the diagnosis result display information transmitted from the second information processing apparatus.

Second disclosure of an information processing apparatus is an information processing apparatus comprising: a processor configured to execute a program; and a storage device configured to store the program, the processor being configured to execute: obtaining processing of obtaining a diagnosis result of both eyes; generation processing of generating diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; and output processing of outputting the diagnosis result display information generated by the generation processing.

Third disclosure of an information processing apparatus is an information processing apparatus comprising: a processor configured to execute a program; and a storage device configured to store the program, the processor being configured to execute: first display control processing of displaying diagnosis result display information on a display screen, the diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; detection processing of detecting an operation in a display region of a subject eye image of a specific eye which is the right eye or the left eye; obtaining processing of obtaining a chronological subject eye image data group of the specific eye; and second display control processing of displaying the chronological subject eye image group of the specific eye in the display region based on the operation detected by the detection processing and the chronological subject eye image data group of the specific eye obtained by the obtaining processing.

Fourth disclosure of an information processing apparatus is an information processing apparatus comprising: a processor configured to execute a program; and a storage device configured to store the program, the processor being configured to execute: first display control processing of displaying diagnosis result display information on a display screen, the diagnosis result display information including a scale indicating a plurality of degrees of progresses related to a symptom and aligned in order of the progresses, a first index indicating a degree of progress of a right eye based on a diagnosis result of the right eye, and a second index indicating a degree of progress of a left eye based on a diagnosis result of the left eye, detection processing of detecting an operation in an alignment direction of the plurality of degrees of progresses, obtaining processing of obtaining a chronological subject eye image data group of a specific eye of the right eye or the left eye, and second display control processing of moving the plurality of degrees of progresses based on the operation in the alignment direction detected by the detection processing, and displaying a subject eye image of the specific eye associated with a moved degree of progress indicated by a specific index of the first index or the second index based on the chronological subject eye image data group of the specific eye obtained by the obtaining processing.

Fifth disclosure of an information processing apparatus is an information processing apparatus comprising: a processor configured to execute a program; and a storage device configured to store the program, the processor being configured to execute: first display control processing of displaying diagnosis result display information on a display screen, the diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; detection processing of detecting an operation in a direction from a first display region of a subject eye image of one eye of the right eye and the left eye to a second display region of a subject eye image of other one eye; and second display control processing of, when the operation is detected by the detection processing, displaying the subject eye image of the one eye in the second display region and displaying the subject eye image of the other eye in the first display region.

Sixth disclosure of a computer-readable recording medium is a computer-readable recording medium having recorded thereon an information processing program causing a processor to execute: obtaining processing of obtaining a diagnosis result of both eyes; generation processing of generating diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; and output processing of outputting the diagnosis result information generated by the generation processing.

Seventh disclosure of a computer-readable recording medium is a computer-readable recording medium having recorded thereon an information processing program causing a processor to execute: first display control processing of displaying diagnosis result display information on a display screen, the diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; detection processing of detecting an operation in a display region of a subject eye image of a specific eye of the right eye or the left eye; obtaining processing of obtaining a chronological subject eye image data group of the specific eye; and second display control processing of displaying the chronological subject eye image group of the specific eye in the display region based on the operation detected by the detection processing and the chronological subject eye image data group of the specific eye obtained by the obtaining processing.

Eighth disclosure of a computer-readable recording medium is a computer-readable recording medium having recorded thereon an information processing program causing a processor to execute: first display control processing of displaying diagnosis result display information on a display screen, the diagnosis result display information including a scale indicating a plurality of degrees of progresses related to a symptom and aligned in order of the progresses, a first index indicating a degree of progress of a right eye based on a diagnosis result of the right eye, and a second index indicating a degree of progress of a left eye based on a diagnosis result of the left eye; detection processing of detecting an operation in an alignment direction of the plurality of degrees of progresses; obtaining processing of obtaining a chronological subject eye image data group of a specific eye which is the right eye or the left eye; and second display control processing of moving the plurality of degrees of progresses based on the operation in the alignment direction detected by the detection processing, and displaying a subject eye image associated with a moved degree of progress indicated by the first index or the second index based on the chronological subject eye image data group of the specific eye obtained by the obtaining processing.

Ninth disclosure of a computer-readable recording medium is a computer-readable recording medium having recorded thereon an information processing program causing a processor to execute: first display control processing of displaying diagnosis result display information on a display screen, the diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; detection processing of detecting an operation in a direction from a first display region of a subject eye image of one eye of the right eye and the left eye to a second display region of a subject eye image of other one eye; and second display control processing of, when the operation is detected by the detection processing, displaying the subject eye image of the one eye in the second display region and displaying the subject eye image of the other eye in the first display region.

Tenth disclosure of an information processing method is an information processing method executed by an information processing apparatus comprising a processor configured to execute a program; and a storage device configured to store the program executed by the processor, the information processing method comprising: obtaining processing of obtaining a diagnosis result of both eyes; generation processing of generating diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; and output processing of outputting the diagnosis result display information generated by the generation processing.

Eleventh disclosure of an information processing method is an information processing method executed by an information processing apparatus comprising a processor configured to execute a program; and a storage device configured to store the program executed by the processor, the information processing method comprising: first display control processing of displaying diagnosis result display information on a display screen, the diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; detection processing of detecting an operation in a display region of a subject eye image of a specific eye of the right eye or the left eye; obtaining processing of obtaining a chronological subject eye image data group of the specific eye; and second display control processing of displaying the chronological subject eye image group of the specific eye in the display region based on the operation detected by the detection processing and the chronological subject eye image data group of the specific eye obtained by the obtaining processing.

Twelfth disclosure of an information processing method is an information processing method executed by an information processing apparatus comprising a processor configured to execute a program; and a storage device configured to store the program executed by the processor, the information processing method comprising: first display control processing of displaying diagnosis result display information on a display screen, the diagnosis result display information including a scale indicating a plurality of degrees of progresses related to a symptom and aligned in order of the progresses, a first index indicating a degree of progress of a right eye based on a diagnosis result of the right eye, and a second index indicating a degree of progress of a left eye based on a diagnosis result of the left eye, detection processing of detecting an operation in an alignment direction of the plurality of degrees of progresses, obtaining processing of obtaining a chronological subject eye image data group of a specific eye of the right eye or the left eye, and second display control processing of moving the plurality of degrees of progresses based on the operation in the alignment direction detected by the detection processing, and displaying a subject eye image of the specific eye associated with a moved degree of progress indicated by a specific index of the first index or the second index based on the chronological subject eye image data group of the specific eye obtained by the obtaining processing.

Thirteenth disclosure of an information processing method is an information processing method executed by an information processing apparatus comprising a processor configured to execute a program; and a storage device configured to store the program executed by the processor, the information processing method comprising: first display control processing of displaying diagnosis result display information on a display screen, the diagnosis result display information including a scale indicating a plurality of degrees of progresses related to a symptom, a first index indicating a degree of progress of a right eye based on a diagnosis result of the right eye, and a second index indicating a degree of progress of a left eye based on a diagnosis result of the left eye; detection processing of detecting an operation in a direction from a first display region of a subject eye image of one eye of the right eye and the left eye to a second display region of a subject eye image of other one eye; and second display control processing of, when the operation is detected by the detection processing, displaying the subject eye image of the one eye in the second display region and displaying the subject eye image of the other eye in the first display region.

### BRIEF DESCRIPTION OF THE DRAWINGS

FIG. 1 is an explanatory diagram showing a display screen example 1 indicating a diagnosis result of fundus images.

FIG. 2 is an explanatory diagram showing a display screen example 2 indicating a diagnosis result of fundus images.

FIG. 3 is an explanatory diagram showing an operation example of a GUI (Graphical User Interface) on the display screen shown in FIG. 1.

FIG. 4 is an explanatory diagram 1 showing an example of the moving operation of fundus images.

FIG. 5 is an explanatory diagram 2 showing an example of the moving operation of fundus images.

FIG. 6 is an explanatory diagram 1 showing an example of the moving operation of the marker.

FIG. 7 is an explanatory diagram 2 showing an example of the moving operation of the marker.

FIG. 8 is an explanatory diagram 3 showing an example of the moving operation of the marker.

FIG. 9 is an explanatory diagram 4 showing an example of the moving operation of the marker.

FIG. 10 is an explanatory diagram showing an example of the display screen after the left/right switching operation.

FIG. 11 is an explanatory diagram showing a system configuration example of an information processing system.

FIG. 12 is a block diagram illustrating a hardware configuration example of a computer.

FIG. 13 is an explanatory diagram showing an example of the contents stored in the patient information DB.

FIG. 14 is an explanatory diagram showing an operation sequence example of the information processing system.

FIG. 15 is a flowchart showing an example of a detailed processing procedure of the display processing (step S1415) performed by the terminal and shown in FIG. 14.

FIG. 16 is a flowchart showing an example of a detailed processing procedure of the display change processing (step S1504) based on the detection result shown in FIG. 15.

FIG. 17 is a flowchart showing an example of a detailed processing procedure of the intra-fundus image display region change processing (step S1602) shown in FIG. 16.

FIG. 18 is a flowchart showing an example of a detailed processing procedure of the marker position change processing (step S1603) shown in FIG. 16.

FIG. 19 is a flowchart showing an example of a detailed processing procedure of the change processing of the scale (step S1604) shown in FIG. 16.

FIG. 20 is a flowchart showing an example of a detailed processing procedure of the left/right switching processing (step S1605) shown in FIG. 16.

FIG. 21 is a block diagram showing a functional configuration example of the information processing system.

### DISPLAY SCREEN AND SCREEN OPERATION

In the following embodiments, a description will be given on an example of a display screen and an operation example of the display screen, the display screen indicating a diagnosis result of fundus images as an example of subject eye images of both subject eyes of a patient. It is noted that for each sign shown in the figures below, a sign with “R” or “R#” (where “#” is a number) attached at its end indicates that the data is related to the right subject eye. Further, a sign with “L” or “L#” (where “#” is a number) attached at its end indicates that the data is related to the left subject eye. When the left and right are not distinguished from each other, “R”, “R#”, “L”, and “L#” may be omitted. Although the terms “image data” and “image” are synonymous, when the “image data” is output to a display device with a display screen, the “image” is displayed on the display screen. It is noted that the “image” and “image data” are denoted by the same sign.

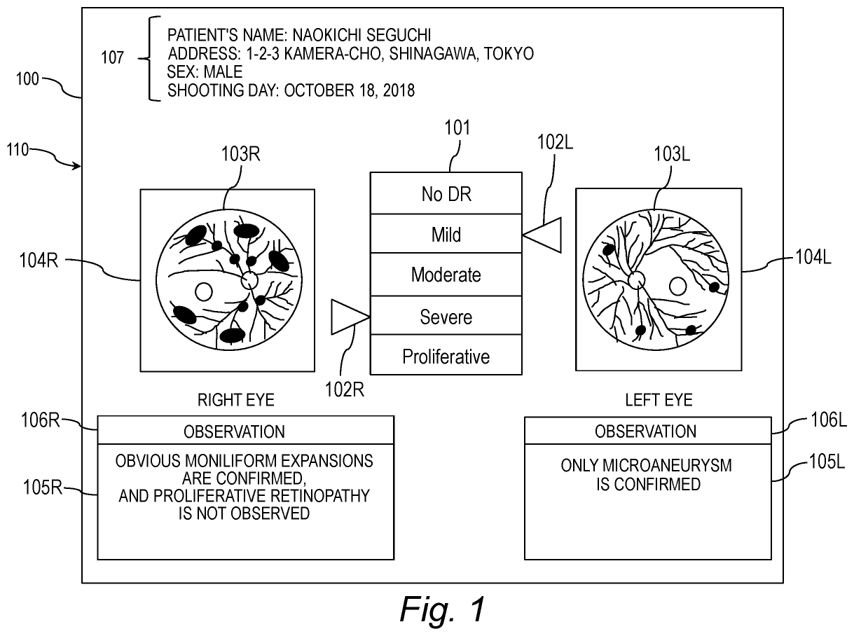

FIG. 1 is an explanatory diagram showing a display screen example 1 indicating a diagnosis result of fundus images. A display screen 110 is displayed on a display device of a computer. The display screen 110 displays diagnosis result display information 100 that includes a scale 101, a marker 102, a fundus image 103, a fundus image display region 104, an observation 105, an observation display region 106, and patient information 107.

The scale 101 indicates a plurality of degrees of progress related to a symptom. Here, a plurality of degrees of progress related to the symptoms of diabetic retinopathy are described as an example. For example, according to the International Severity Classification, the degrees of progress are classified into “No DR”, “Mild”, “Moderate”, “Severe” and “Proliferative”. In the scale 101, the plurality of degrees of progress are aligned in order from “No DR” to “Proliferative”, from the top.

The “No DR” indicates no apparent diabetic retinopathy. The “Mild” indicates mild nonproliferative diabetic retinopathy. The “Moderate” indicates moderate nonproliferative diabetic retinopathy. The “Severe” indicates severe nonproliferative diabetic retinopathy. The “Proliferative” indicates proliferative diabetic retinopathy. In addition to the International Severity Classification, the plurality of degrees of progress may be classified according to other classifications such as the Modified Davis Classification, the New Fukuda Classification, and the ETDRS Classification.

The scale 101 is disposed between fundus images 103R and 103L. The plurality of degrees of progress are aligned, for example, along the longitudinal direction in order of the progresses. Thus, the scale 101 does not need to be disposed for each of the fundus images 103R and 103L. In this way, the scale 101 is shared between the two fundus images 103R and 103L, thus improving the visibility of the display screen 110. Moreover, the other information in the display screen 110 can be enlarged accordingly. It can also be said that the scale 101 is disposed between a right-eye fundus image display region 104R and a left-eye fundus image display region 104L.

The marker 102 is an index specifying which one of the plurality of degrees of progress the degree of progress related to the symptom shown in the fundus image 103 corresponds to. A marker 102R indicates which one of the plurality of degrees of progress the degree of progress related to the symptom of the right eye shown in the fundus image 103R corresponds to. The marker 102R is disposed between the scale 101 and the fundus image 103R of the right eye. In FIG. 1, the marker 102R indicates the “Severe”. Therefore, the symptom of the right eye, shown in the fundus image 103R, is attributed to severe nonproliferative diabetic retinopathy. It can also be said that the marker 102R is disposed between the scale 101 and the right-eye fundus image display region 104R.

A marker 102L indicates which one of the plurality of degrees of progress the degree of progress related to the symptom of the left-eye shown in the fundus image 103L corresponds to. The marker 102L is disposed between the scale 101 and the fundus image 103L of the left eye. In FIG. 1, the marker 102L indicates the “Mild”. Therefore, the symptom of the left eye, shown in the fundus image 103L, is attributed to moderate nonproliferative diabetic retinopathy. Thus, users such as physicians can easily identify the symptom from the fundus image 103. It can also be said that the marker 102L is disposed between the scale 101 and the left-eye fundus image display region 104L.

The fundus image 103 is a subject eye image provided by shooting a subject eye of a patient. The fundus image display region 104 is a region where the fundus image 103 is displayed. The observation 105 is composed of a string of characters that indicates opinions and thoughts about the subject eye. The observation 105 is stored in a storage device 1202 while being linked to the degree of progress of the symptom. Therefore, an observation 105R of the right eye is an observation associated with the severe nonproliferative diabetic retinopathy, while an observation 105L of the left eye is an observation associated with the moderate nonproliferative diabetic retinopathy.

The observation display region 106 is a region where the observation 105 is displayed. The observation 105R of the right eye is displayed in an observation display region 106R, while the observation 105L of the left eye is displayed in an observation display region 106L. The patient information 107 is personal information on the patient whose subject eye is shot.

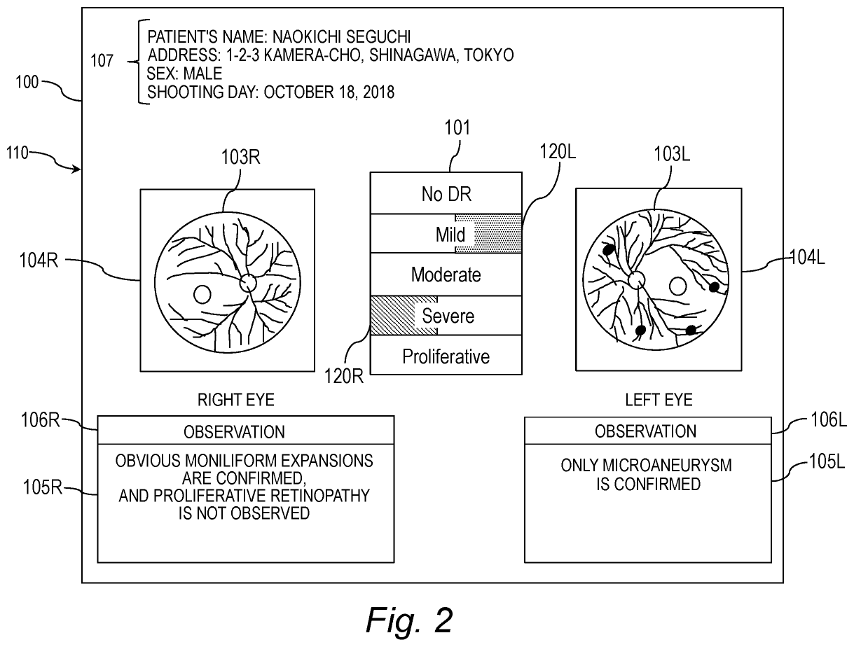

FIG. 2 is an explanatory diagram showing a display screen example 2 indicating a diagnosis result of fundus images. FIG. 2 shows another example of indexes. In FIG. 2, on the display screen 110, the marker 120 specifies the degree of progress related to the symptom shown in the fundus image, by means of highlighted color (indicated by hatching) and a highlighted position within the region for the degree of progresses (near the fundus image). For example, for a fundus image of the right eye, the marker 120R specifies that the symptom is attributed to the severe nonproliferative diabetic retinopathy by highlighting the left half of a region for the degree of progress “Severe” with hatching.

Likewise, for a fundus image of the left eye, a marker 120L specifies that the symptom is attributed to mild nonproliferative diabetic retinopathy by highlighting the right half of a region for the degree of progress “Mild” with hatching. This allows a user to intuitively identify the symptom shown in the fundus image. If there is a space between the scale 101 and the fundus image, the marker 102 can be disposed in the space as shown in FIG. 1. On the other hand, in the case of a layout that does not have any space, this marker 120 is effective. It is noted that in FIG. 2, the marker 102 shown in FIG. 1 may be displayed.

### Operation Example of GUI on Display Screen 110

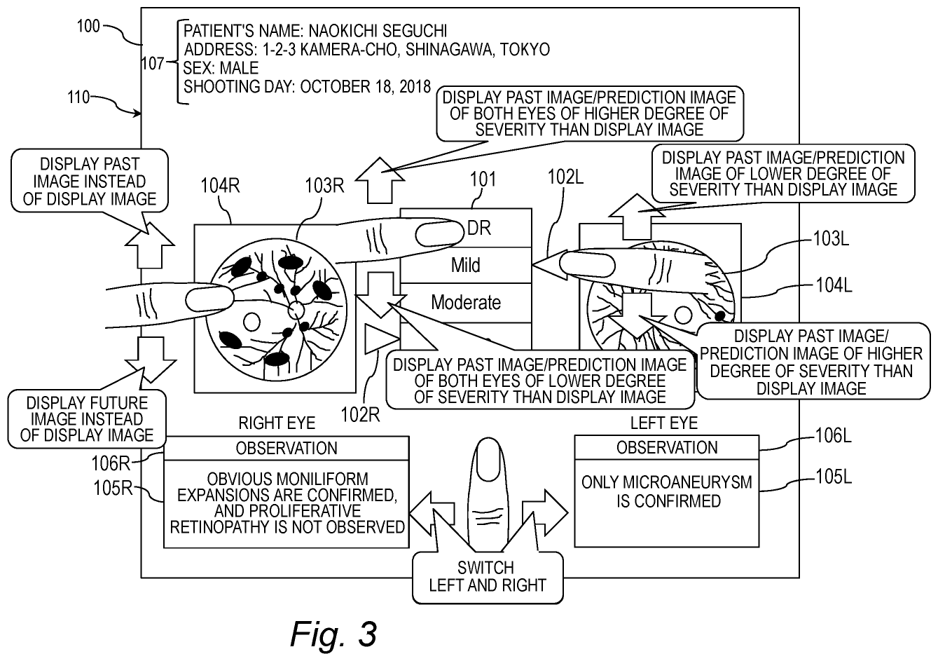

FIG. 3 is an explanatory diagram showing an operation example of a GUI (Graphical User Interface) on the display screen 110 shown in FIG. 1. FIG. 3 indicates operation points of the GUI that can be operated by a user, for example, with his/her finger (may be operated with a mouse or pen). It is noted that even on the display screen 110 shown in FIG. 2, the same operation can be performed.

### [Moving Operation of Fundus Image 103]

The user can place the finger within the fundus image display region 104 to perform a moving operation (so-called sliding) of the finger in a certain direction on the display screen 110. Thus, the fundus image 103, which is an object within the fundus image display region 104, moves in the certain direction. Although in FIG. 3, the fundus image 103R, which is an object within the fundus image display region 104R, is described as an example, the same can be applied to the fundus image 103L, which is an object in the fundus image display region 104L.

The moving operation in the certain direction is, for example, swiping or flicking. For the swiping, the user places the finger at any position within the fundus image display region 104R (even a position where the fundus image 103R is not present) and slides the finger in the certain direction. For the flicking, the user places the finger on the fundus image 103R and slides the finger in the certain direction.

The certain direction is, for example, the upper/downward direction (may be the left/right direction). For example, by performing the moving operation in the upward direction, the fundus image 103R which is being displayed within the fundus image display region 104R moves upward and then disappears from the fundus image display region 104R, and concurrently another fundus image 103R appears from a lower end of the fundus image display region 104R.

The other fundus image 103R is, for example, a subject eye image of the right eye of the same patient shot at a different time from the current fundus image 103R. For example, for the moving operation in the upward direction, the other fundus image 103R is a subject eye image of the right eye of the same patient shot in the past, compared to the current fundus image 103R. For the moving operation in the downward direction, the other fundus image 103R is a subject eye image of the right eye of the same patient to be shot after (in the future) shooting the current fundus image 103R.

Alternatively, the moving operation may be an operation of tapping either one of both ends in the certain direction at borders of the fundus image display region 104. For example, by performing the tapping operation at the upper end of the fundus image display region 104R, the fundus image 103R which is being displayed within the fundus image display region 104R moves upward and then disappears from the fundus image display region 104R, while another fundus image 103R appears from the lower end of the fundus image display region 104R. Likewise, by performing the tapping operation at the lower end of the fundus image display region 104R, the fundus image 103R which is being displayed within the fundus image display region 104R moves downward and then disappears from the fundus image display region 104R, while another fundus image 103R appears from the upper end of the fundus image display region 104R. Consequently, a series of fundus images shot at different shooting times can be displayed in sequence.

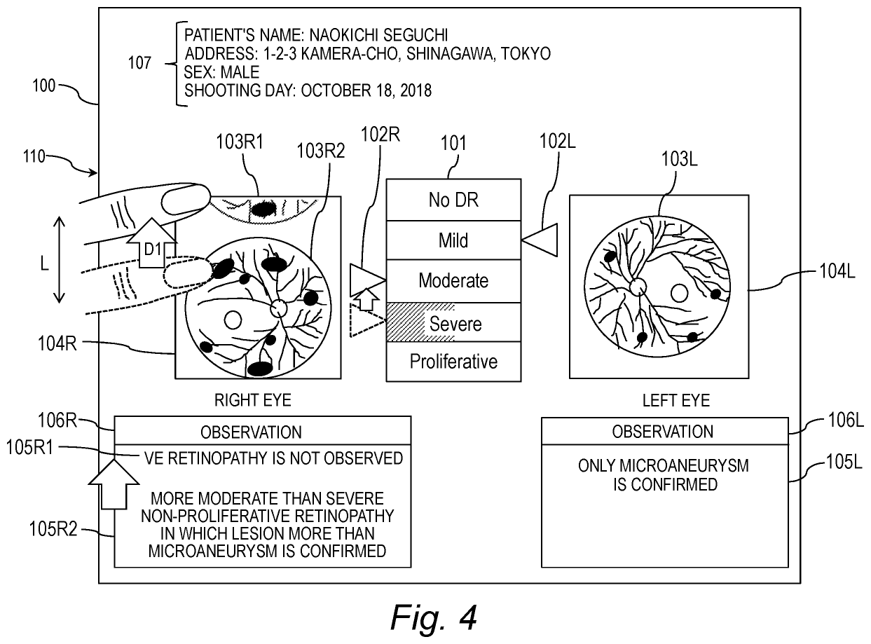

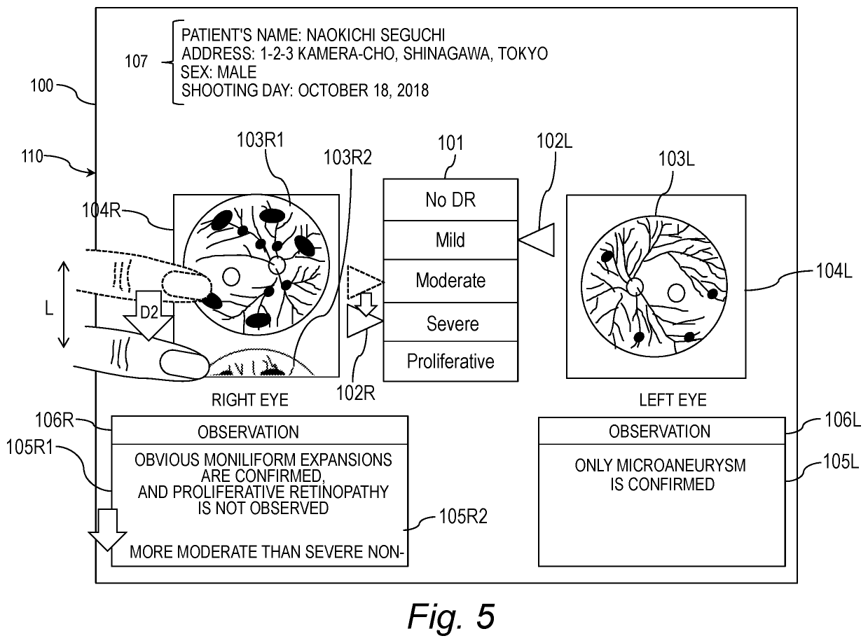

FIGS. 4 and 5 are explanatory diagrams showing an example of the moving operation of fundus images. FIG. 4 shows the moving operation in the upward direction D1, while FIG. 5 shows the moving operation in the downward direction D2. In FIGS. 4 and 5, an example of the moving operation of the fundus image 103R of the right eye is described, but an example of the moving operation of the fundus image 103L of the left eye is the same as that of the right eye and thus its description will be omitted.

In FIG. 4, the user places the finger on the fundus image display region 104R and slides the finger by a distance L in the upward direction D1. Thus, the fundus image 103R1 for the degree of progress “Severe” displayed in the fundus image display region 104R moves in the upward direction D1 according to the upward direction D1 and the operation distance L and then disappears at the upper end of the fundus image display region 104R. In addition, the fundus image 103R2 for the degree of progress “Moderate” not displayed in the fundus image display region 104R moves in the upward direction D1 according to the upward direction D1 and the operation distance L and then appears from the lower end of the fundus image display region 104R.

In conjunction with the moving operation in the upward direction D1, the marker 102R moves from the position indicating the “Severe” to the position indicating the “Moderate”. Likewise, in conjunction with the moving operation in the upward direction D1, an observation 105R1 displayed in the observation display region 106R moves in the upward direction D1 and then disappears at the upper end of the observation display region 106R.

In conjunction with the moving operation in the upward direction D1, an observation 105R2 not displayed in the observation display region 106R moves in the upward direction D1 and then appears from the lower end of the observation display region 106R. The left half of the degree of progress “Severe” may be highlighted (hatched in FIG. 4) so that the position of the marker 102R indicating the degree of progress “Severe” before the movement can be understood. In this way, after the movement of the marker 102, the user can visually recognize which one of the degrees of progress the degree of progress indicated before the movement of the marker 102 corresponds to.

FIG. 5 shows an example of returning the diagnosis result display information to the state before the movement shown in FIG. 4 by performing the moving operation in the upward direction D1 shown in FIG. 4, followed by the other moving operation in the downward direction D2. In FIG. 5, the user places the finger on the fundus image display region 104R and then slides the finger by the distance L in the downward direction D2. Thus, the fundus image 103R2 for the degree of progress “Moderate” displayed in the fundus image display region 104R moves in the downward direction D2 according to the downward direction D2 and the distance L and then disappears at the lower end of the fundus image display region 104R. In addition, the fundus image 103R1 for the degree of progress “Severe” not displayed in the fundus image display region 104R at the moment moves in the downward direction D2 according to the downward direction D2 and the distance L and then appears from the upper end of the fundus image display region 104R.

In conjunction with the moving operation in the downward direction D2, the marker 102R moves from the position indicating the “Moderate” to the position indicating the “Severe”. Likewise, in conjunction with the moving operation in the downward direction D2, the observation 105R2 displayed in the observation display region 106R moves in the downward direction D2 and then disappears at the lower end of the observation display region 106R.

In conjunction with the moving operation in the downward direction D2, the observation 105R1 not displayed in the observation display region 106R moves in the downward direction D2 and then appears from the upper end of the observation display region 106R. Since the diagnosis result display information 100 returns to the state before the movement shown in FIG. 4, when the left half of the degree of progress “Severe” is highlighted (hatched in FIG. 4), this highlighting is terminated. It is noted that in a case where there is no fundus image 103 of a movement destination in FIGS. 4 and 5, the fundus image 103 does not move in the upper/downward direction.

### [Moving Operation of Marker 102]

Returning to FIG. 3, the user can place the finger on the marker 102, which is an object on the display screen 110 to perform a moving operation of the finger in a certain direction on the display screen 110. Thus, the marker 102 moves in the certain direction. Although in FIG. 3, the marker 102L, which is an object located between the scale 101 and the fundus image display region 104L, is described as an example, the same can be applied to the marker 102R, which is an object located between the scale 101 and the fundus image display region 104R.

The moving operation of the marker 102 in the certain direction is, for example, flicking. The certain direction in which the marker 102 is moved is, for example, an alignment direction of the degrees of progress, i.e., the upper/downward direction. For example, by performing the moving operation in the upward direction D1, the marker 102L moves upward and indicates the degree of progress with lower severity. Thus, in conjunction with the movement of the marker 102L, another fundus image 103L different from the fundus image 103L being displayed is displayed in the fundus image display region 104L.

The other fundus image 103L is, for example, a past fundus image 103L or predicted fundus image of the same patient with lower severity than the fundus image 103L being displayed. The predicted fundus image is a fundus image predicted according to the degree of progress indicated by the moved marker 102L, based on the fundus image 103L being displayed. The computer displaying the predicted fundus image may generate image data about the predicted fundus image or may receive image data about the predicted fundus image from another computer that is communicably connected with this computer.

By performing the moving operation in the downward direction, the marker 102L moves downward and indicates the degree of progress with higher severity. Thus, in conjunction with the movement of the marker 102L, another fundus image 103L, which is different from the fundus image 103L being displayed, is displayed within the fundus image display region 104L. The other fundus image 103L is, for example, a past fundus image or predicted fundus image of the same patient with higher severity than the fundus image 103L being displayed.

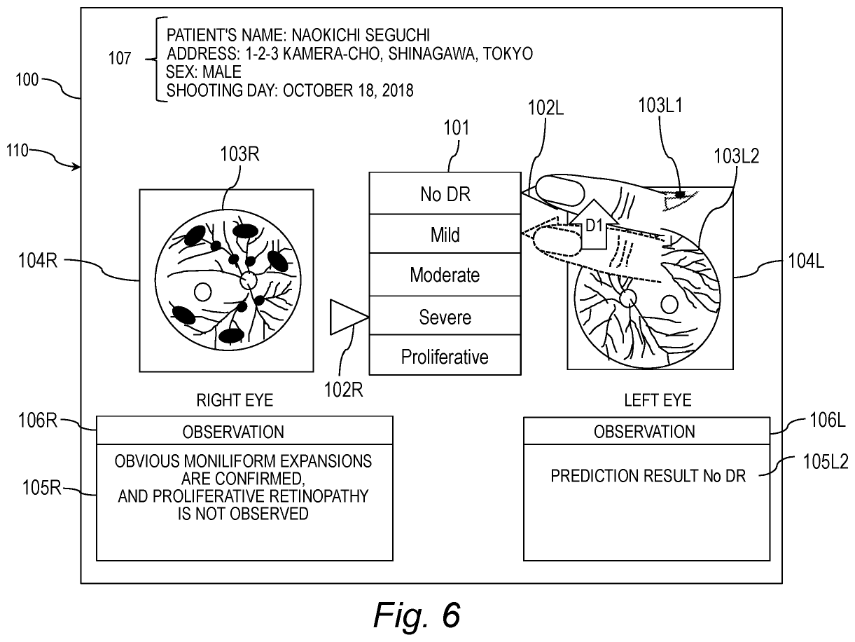

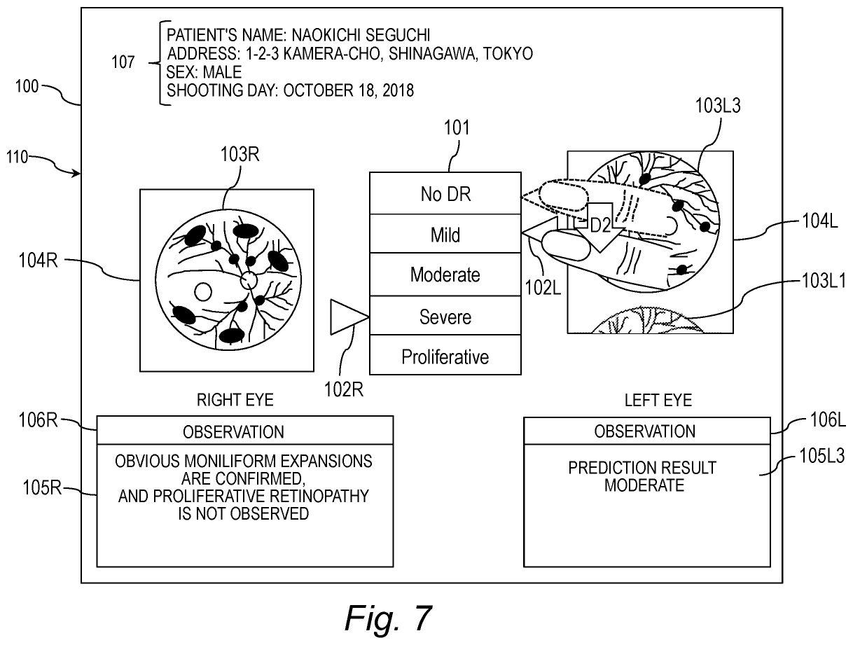

FIGS. 6 and 7 are explanatory diagrams showing an example of the moving operation of the marker. FIG. 6 shows the moving operation in the upward direction D1, while FIG. 7 shows the moving operation in the downward direction D2. In FIGS. 6 and 7, an example of the moving operation of the marker 102L is described, but an example of the moving operation of the marker 102R is the same as that of the marker 102L and thus its description will be omitted.

In FIG. 6, the user places the finger on the marker 102L and slides the finger in the upward direction D1. Thus, the marker 102L moves from the position indicating the degree of progress “Mild” to the position indicating the degree of progress “No DR” according to the moving operation in the upward direction D1.

Thus, the fundus image 103L1 for the degree of progress “Mild” displayed in the fundus image display region 104L moves in the upward direction D1 and then disappears at the upper end of the fundus image display region 104L. In addition, the fundus image 103L2 for the degree of progress “No DR” not displayed in the fundus image display region 104 moves in the upward direction D1 according to the moving operation in the upward direction D1 and then appears from the lower end of the fundus image display region 104L.

In a case where there is no fundus image 103L2, the fundus image 103L1 remains displayed in the fundus image display region 104L, and the marker 102L does not move to the position indicating “No DR.” Alternatively, in a case where there is no fundus image 103L2, a predicted fundus image for the degree of progress “No DR” to be indicated by the moved marker may be displayed in the fundus image display region 104L.

In conjunction with the moving operation in the upward direction D1, an observation 105L2 not displayed in the observation display region 106L moves in the upward direction D1 to appear from the lower end of the observation display region 106L and is then displayed in the observation display region 106L. In a case where there is no fundus image 103L2, the observation of the fundus image 103L1 remains displayed in the observation display region 106L. Alternatively, in a case where there is no fundus image 103L2, an observation 105L2 of a predicted fundus image for the degree of progress “No DR” to be indicated by the moved marker 102L may be displayed in the observation display region 106L.

In FIG. 7, the user places the finger on the marker 102L and slides the finger in the downward direction D2. Thus, the marker 102L moves from the position indicating the degree of progress “Mild” to the position indicating the degree of progress “Moderate” according to the moving operation in the downward direction D2.

The fundus image 103L1 for the degree of progress “Mild” displayed in the fundus image display region 104L moves in the downward direction D2 and then disappears at the lower end of the fundus image display region 104L. In addition, the fundus image 103L3 for the degree of progress “Moderate” not displayed in the fundus image display region 104L moves in the downward direction D2 according to the moving operation in the downward direction D2 and then appears from the upper end of the fundus image display region 104L.

In a case where there is no fundus image 103L3, the fundus image 103L1 remains displayed in the fundus image display region 104L, and the marker 102L does not move to the position indicating “Moderate”. Alternatively, in a case where there is no fundus image 103L3, a predicted fundus image for the degree of progress “Moderate” to be indicated by the moved marker may be displayed in the fundus image display region 104L.

In conjunction with the moving operation in the downward direction D2, the observation 105L3 not displayed in the observation display region 106L moves in the downward direction D2 to appear from the upper end of the observation display region 106L and is then displayed in the observation display region 106L. In a case where there is no fundus image 103L3, the observation of the fundus image 103L1 remains displayed in the observation display region 106L. Alternatively, in a case where there is no fundus image 103L3, an observation 105L3 of a predicted fundus image for the degree of progress “Moderate” to be indicated by the moved marker 102L may be displayed in the observation display region 106L.

### [Moving Operation of Degree of Progress]

Returning to FIG. 3, the user can place the finger on the scale 101 and perform an operation of moving the degree of progress on the display screen 110 in the certain direction. Thus, the plurality of degrees of progress are scrolled in the certain direction. The operation of moving the degrees of progress in the certain direction is, for example, flicking. The certain direction in which the degrees of progress are moved is, for example, the alignment direction of the degrees of progress, i.e., the upper/downward direction.

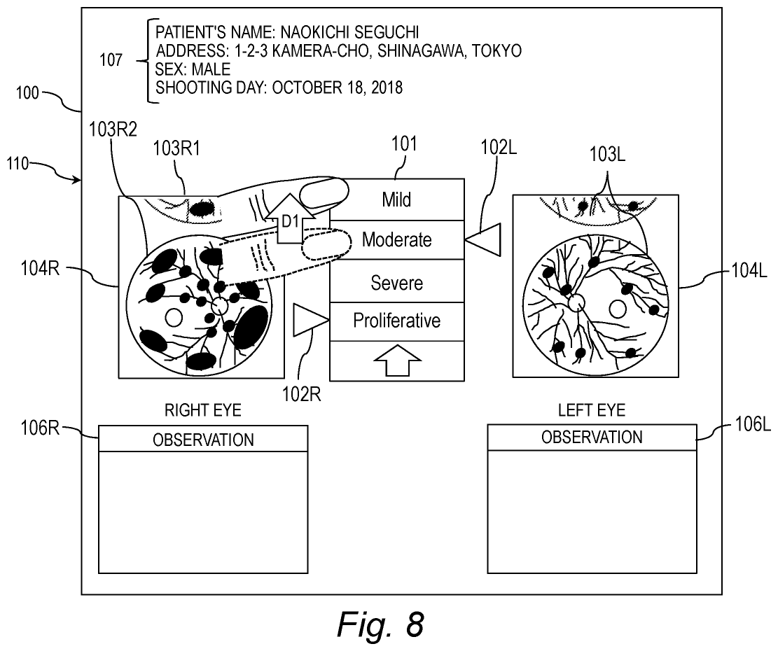

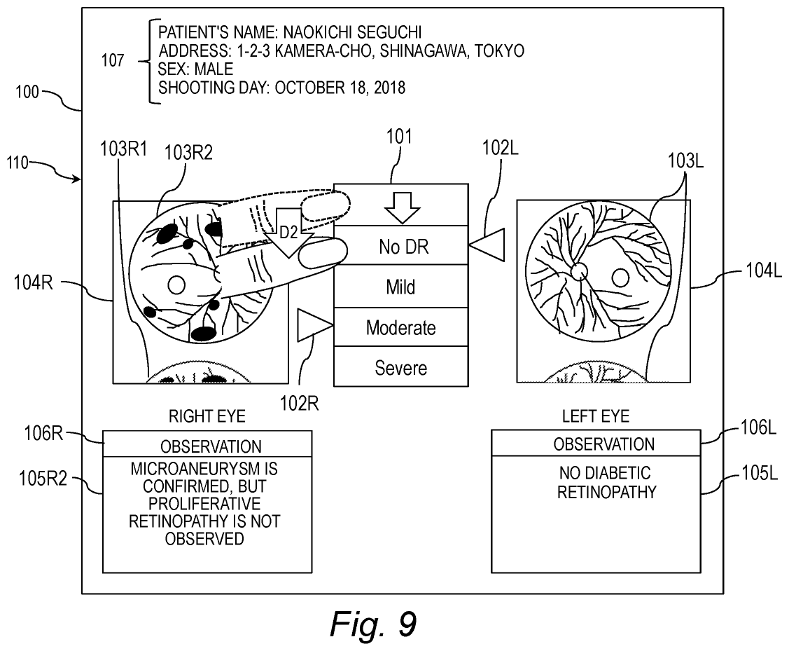

FIGS. 8 and 9 are explanatory diagrams showing an example of the moving operation of the marker 102. FIG. 8 shows the moving operation in the upward direction D1, while FIG. 9 shows the moving operation in the downward direction D2. In FIG. 8, by performing the moving operation in the upward direction, the plurality of degrees of progress are scrolled upward while the position of the marker 102 is not changed, so that the marker 102 indicates the degree of progress with higher severity. Thus, in conjunction with the scrolling, another fundus image 103 different from the fundus image 103 being displayed is displayed within the fundus image display region 104. The other fundus image 103 is, for example, a past fundus image or predicted fundus image of the same patient with higher severity than the fundus image 103 being displayed.

In FIG. 9, by performing the moving operation in the downward direction, the plurality of degrees of progress are scrolled downward while the position of the marker 102 is not changed, so that the marker 102 indicates the degree of progress with lower severity. Thus, in conjunction with the scrolling, another fundus image 103 different from the fundus image 103 being displayed is displayed within the fundus image display region 104. The other fundus image 103 is, for example, a past fundus image or predicted fundus image of the same patient with lower severity than the fundus image 103 being displayed.

### [Left/Right Switching Operation]

Returning to FIG. 3, the user can place the finger on the display screen 110 and perform an operation of sliding the finger in a predetermined direction on the display screen 110. This switches the display positions of the fundus image, marker 102, and observation 105 on the right side with those on the left and vice versa. The operation of sliding the finger in the certain direction is, for example, swiping or flicking. The predetermined direction is a direction different from the certain direction described above, for example, the left/right direction. In FIG. 3, the fundus image 103R of the right eye, the observation 105R, and a string of characters of “right eye” are displayed on the left side as viewed facing the screen, while the fundus image 103L of the left eye, the observation 105L, and a string of characters of “left eye” are displayed on the right side as viewed facing the screen.

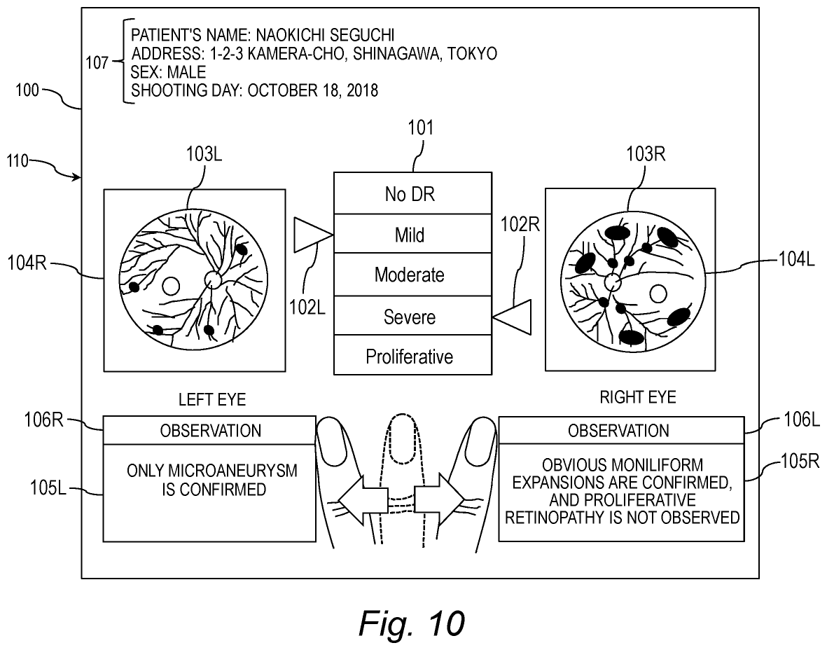

FIG. 10 is an explanatory diagram showing an example of the display screen after the left/right switching operation. When performing the left/right switching operation from the state of FIG. 3, as shown in FIG. 10, the fundus image 103L of the left eye, the observation 105L, and a string of characters of “left eye” are displayed on the left side as viewed facing the screen, while the fundus image 103R of the right eye, the observation 105R, and a string of characters of “right eye” are displayed on the right side as viewed facing the screen. This is a convenient operation when the user intends to flip the display positions of the right and left eyes from side to side depending on the positional relationship between the patient and the display.

### System Configuration Example of Information Processing System

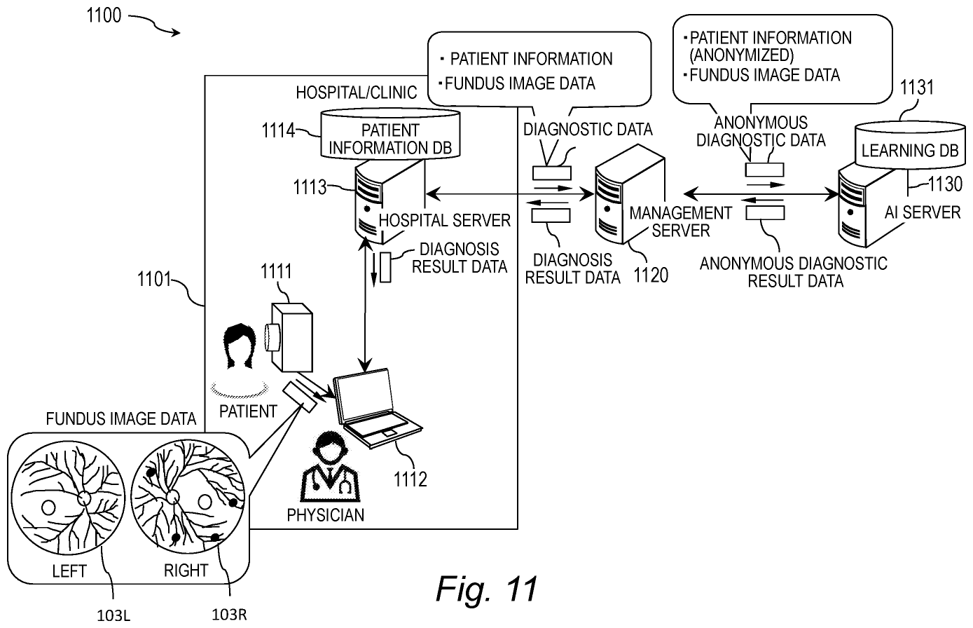

FIG. 11 is an explanatory diagram showing a system configuration example of an information processing system. An information processing system 1100 includes an in-hospital system 1101, an administrative server 1120, and an AI (Artificial Intelligence) server 1130. The in-hospital system 1101 and the administrative server 1120 are communicably connected with each other. The administrative server 1120 and the AI server are communicably connected with each other.

The in-hospital system 1101 is provided, for example, in hospitals or clinics (for example, an ophthalmologist, an internist, a diabetologist, and the like). The in-hospital system 1101 includes an ophthalmic device 1111, a terminal 1112, and an in-hospital server 1113. The ophthalmic device 1111 is communicably connected with the terminal 1112. The ophthalmic device 1111 is, for example, a fundus camera, or a scanning laser ophtalmoscope or optical coherency tomography, which scans a subject eye with a laser beam to generate an image based on reflected light from the fundus. The ophthalmic device 1111 generates fundus image data 103 of the subject eye. The ophthalmic device 1111 transmits the generated fundus image data 103 to the terminal 1112. The fundus image data 103 includes the shooting date.

The terminal 1112 is a computer communicably connected with the ophthalmic device 1111 and the in-hospital server 1113. The terminal 1112 may be communicably connected directly with the administrative server 1120. The terminal 1112 is used by, for example, a physician. The terminal 1112 is, for example, a personal computer or tablet. As shown in FIGS. 1 to 10, the terminal 1112 receives the fundus image data from the ophthalmic device 1111 and displays the fundus image on the display screen 110.

In addition, the terminal 1112 receives patient information 107 and diagnosis result data from the in-hospital server 1113 and displays them on the display screen 110. The diagnosis result data includes the diagnosis results, which are the degree of progress and observation 105 for each subject eye shown in FIGS. 1 to 10. It is noted that the fundus image data from the ophthalmic device 1111 as well as the patient information 107 and diagnosis result data from the in-hospital server 1113 are linked to the same ID (for example, patient ID) by the terminal 1112 or the in-hospital server 1113.

The in-hospital server 1113 is a computer communicably connected with the terminal 1112 and the administrative server 1120. The in-hospital server 1113 has the patient information DB 1114. The patient information DB 1114 is a database that stores the patient information 107. The in-hospital server 1113 receives the patient ID and the fundus image data 103 from the terminal 1112. The in-hospital server 1113 stores fundus image data 103 in the patient information DB 1114 in associated with to patient information 107 specified by the patient ID.

The in-hospital server 1113 transmits the diagnostic data to the administrative server 1120. The diagnostic data includes the patient information 107 and the fundus image data 103 of the patient. The in-hospital server 1113 receives the diagnosis result data from the administrative server 1120. The in-hospital server 1113 stores the diagnosis result included in the received diagnosis result data, in the patient information DB 1114.

The administrative server 1120 is a computer communicably connected with the in-hospital server 1113 and the AI server 1130. The administrative server 1120 receives the diagnostic data from the in-hospital server 1113. The administrative server 1120 anonymizes the received diagnostic data. Specifically, for example, the administrative server 1120 issues a new ID (hereinafter anonymous ID) and links it to the patient information 107 in the received diagnostic data. The administrative server 1120 defines anonymous diagnostic data as a combination of the anonymous ID and the fundus image data 103.

Furthermore, the patient ID in the patient information 107 may be used as the anonymous ID. In this case, the anonymous diagnostic data is composed of the patient ID, which is the anonymous ID, and the fundus image data 103. This anonymizes the patient information 107. It is noted that if there is any information of the patient information 107 that does not uniquely specify the patient, the administrative server 1120 may include this information in the anonymous diagnostic data. Examples of the information that does not uniquely specify the patient include the patient's vision, gender, age, and nationality.

Moreover, the administrative server 1120 may encrypt the patient information 107. In this case, the administrative server 1120 transmits, to the AI server 1130, a combination of the encrypted patient information 107 and the fundus image data 103 as the anonymous diagnostic data.

The administrative server 1120 receives anonymous diagnostic result data from the AI server 1130. The anonymous diagnostic result data includes the anonymous ID included in the anonymous diagnostic data and diagnosis results which include the degree of progress and the observation 105 for each subject eye shown in FIGS. 1 to 10. The administrative server 1120 converts the received anonymous diagnosis result data into the diagnosis result data.

Specifically, for example, the administrative server 1120 obtains patient information 107 linked to the anonymous ID included in the received anonymous diagnostic result data and switches the anonymous ID with the obtained patient information 107, thereby generating diagnosis result data that includes the patient information 107 and the diagnosis result.

When the administrative server 1120 transmits, to the AI server 1130, the anonymous diagnostic data which is a combination of the encrypted patient information 107 and the fundus image data 103, the administrative server 1120 receives the anonymous diagnostic result data including the encrypted patient information 107 and the diagnosis result, from the AI server 1130.

In this case, the administrative server 1120 converts the anonymous diagnostic result data into diagnosis result data by decrypting the encrypted patient information 107. In this way, the administrative server 1120 can conceal the patient information 107 by anonymization or encryption and thereby can protect the personal information. Thereafter, the administrative server 1120 transmits the generated diagnosis result data to the in-hospital server 1113.

The AI server 1130 is a computer that executes fundus image diagnosis by AI using learning parameters that have been obtained by machine learning or deep learning. The AI server 1130 learns a combination of the past fundus image data 103 and the degree of progress of the symptom at the fundus as training data and generates learning parameters. The training data set and the learning parameters are stored in a learning DB 1131. The learning DB 1131 stores the observation 105 associated with the degree of progress. Using these learning parameters, the AI server 1130 extracts the features of the fundus image through the use of a convolutional neural network (CNN). Then, the AI server 1130 estimates the symptom shown in the input fundus image, based on the features.

The AI server 1130 receives the anonymous diagnostic data. The AI server 1130 inputs the fundus image data 103 included in the anonymous diagnostic data to a learning model in which the learning parameters are applied to the CNN, and then outputs the degree of progress. The AI server 1130 obtains the observation 105 associated with the output degree of progress, from the learning DB 1131.

The AI server 1130 generates anonymous diagnostic result data that includes the anonymous ID included in the anonymous diagnostic data, the degree of progress output from the learning model, and the observation 105 obtained from the learning DB 1131. The AI server 1130 transmits the generated anonymous diagnostic result data to the administrative server 1120.

### Hardware Configuration Example of a Computer

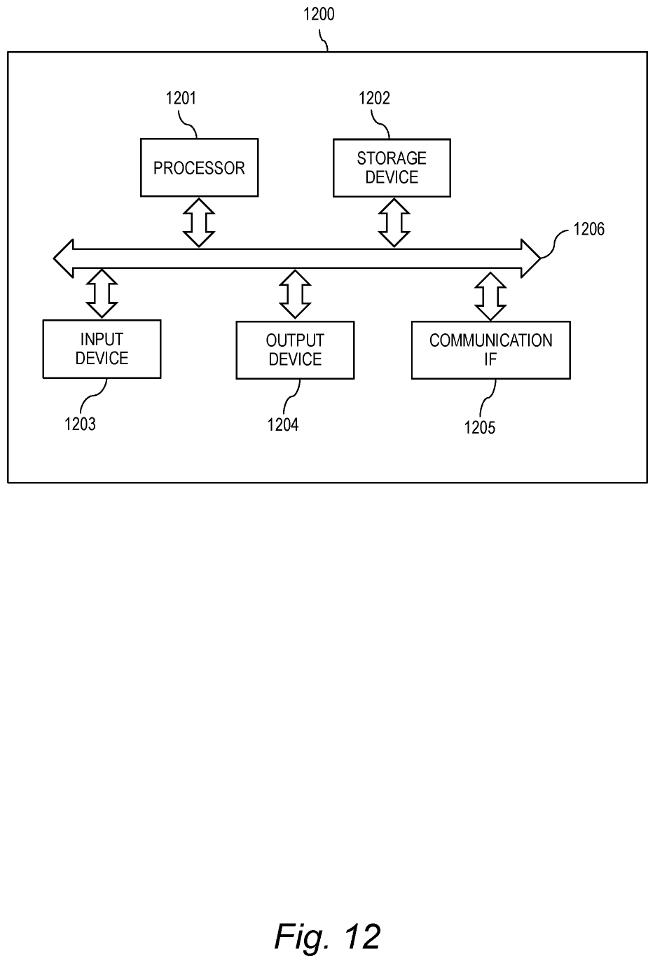

FIG. 12 is a block diagram illustrating a hardware configuration example of a computer (the terminal 1112, the hospital server 1113, the management server 1120, the AI server 1130). The computer 1200 includes a processor 1201, a storage device 1202, an input device 1203, an output device 1204, and a communication interface (communication IF) 1205. The processor 1201, the storage device 1202, the input device 1203, the output device 1204, and the communication IF 1205 are connected to one another by a bus 1206. The processor 1201 controls the computer 1200. The processor 1201 executes various programs. The storage device 1202 serves as a work area of the processor 1201. The storage device 1202 is a non-transitory or temporary recording medium which stores the various programs and data. The storage device 1202 can be, for example, a read-only memory (ROM), a random-access memory (RAM), a hard disk drive (HDD), or a flash memory. The input device 1203 inputs data. The input device 1203 can be, for example, a keyboard, a mouse, a touch panel, a ten-key pad, or a scanner. The output device 1204 outputs data. The output device 1204 can be, for example, a display, a printer or a speaker. The communication IF 1205 couples to a network to transmit and receive data.

### <Patient Information DB 1114>

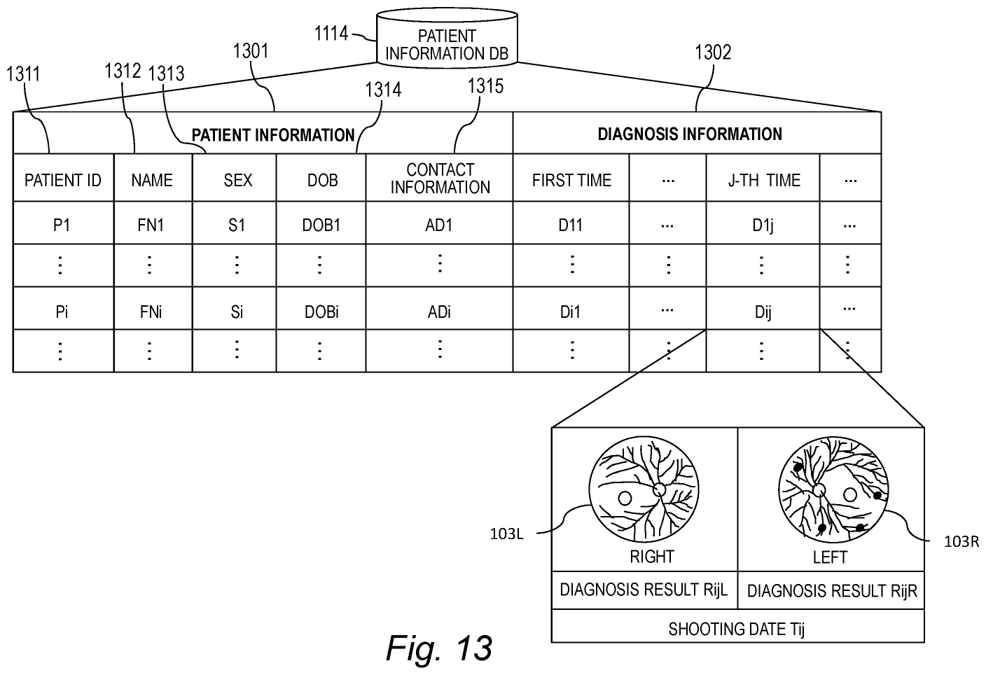

FIG. 13 is an explanatory diagram showing an example of the contents stored in the patient information DB 1114. The patient information DB 1114 includes a patient information field 1301 and a diagnosis information field 1302. The patient information field includes, as sub-fields, a patient ID field 1311, a name field 1312, a gender field 1313, a date of birth field 1314, and a contact field 1315.

The patient information field on the same row covers the patient information 107 of a patient i (i is, for example, an integer of one or more) of the sub-fields 1311 to 1315.

The patient ID field 1311 is a storage region where the patient ID is stored. The patient IDPi is identification information that uniquely specifies the patient i. The name field 1312 is a storage region where a name FNi of the patient i is stored. The gender field 1313 is a storage region where a gender Si of the patient i is stored. The date of birth field 1314 is a storage region where a date of birth DOBi of the patient i is stored. The contact field 1315 is a storage region where contact information ADi of the patient i is stored.

The diagnosis information field 1302 is a storage region where diagnosis information Di1 to Dij including the first to the j-th information on the patient i (j is an integer greater than or equal to one) is stored. The diagnosis information Dij includes the fundus image data 103, the diagnosis result Rij, and the shooting date Tij. The diagnosis result Rij includes the degree of progress and the observation 105, which are provided from the AI server 1130. The shooting date Tij is a date when the fundus image data 103 is generated by shooting the subject eyes in the ophthalmic device 1111.

### Operation Sequence Example of Information Processing System 1100

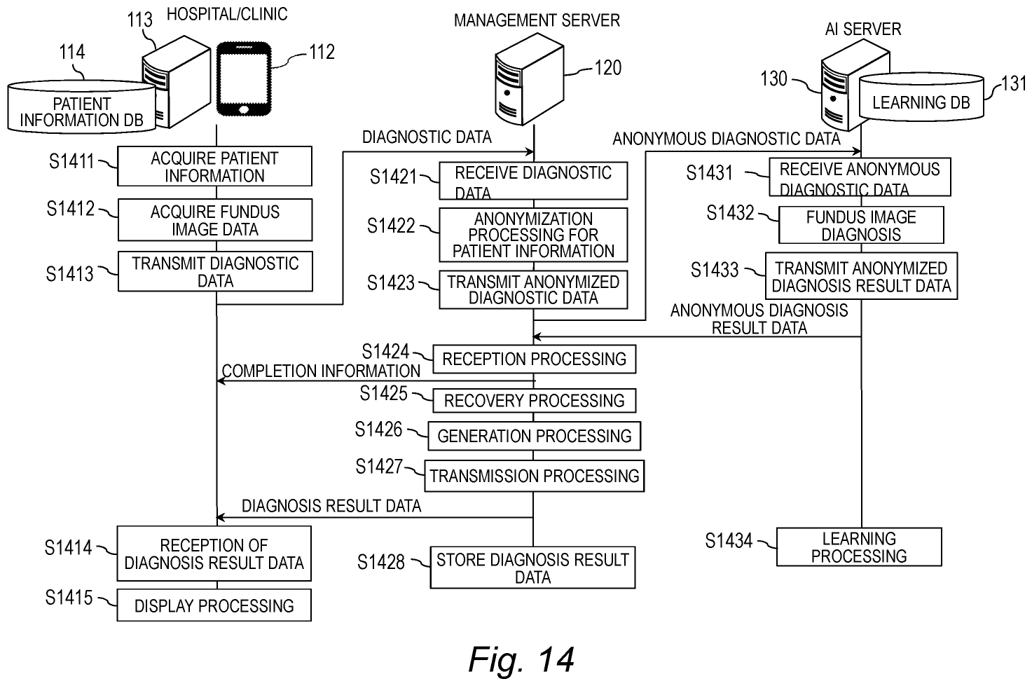

FIG. 14 is an explanatory diagram showing an operation sequence example of the information processing system 1100. The in-hospital server 1113 obtains patient information 107 about a patient, who is a subject, from the patient information DB 1114 (step S1411). The in-hospital server 1113 obtains fundus image data 103 of the subject via the terminal 1112 (step S1412). Then, the in-hospital server 1113 transmits diagnostic data including the patient information 107 obtained in step S1411 and the fundus image data 103 obtained in step S1412, to the administrative server 1120 (step S1413).

The administrative server 1120 receives the diagnosis data when the diagnostic data is transmitted thereto from the in-hospital server 1113 (step S1421). Then, the administrative server 1120 anonymizes the patient information 107 included in the diagnostic data (step S1422). Subsequently, the administrative server 1120 transmits anonymous diagnostic data including an anonymous ID linked to the patient information 107 and the fundus image data 103, to the AI server 1130 (step S1423).

The AI server 1130 receives the anonymous diagnostic data when the anonymous diagnostic data is transmitted thereto from the administrative server 1120 (step S1431). Then, the AI server 1130 executes fundus image diagnosis by inputting the fundus image data 103 included in the anonymous diagnostic data to the learning model (step S1432). Subsequently, the AI server 1130 transmits anonymous diagnostic result data including the anonymous ID, the degree of progress output therefrom by the fundus image diagnosis, and the observation 105 associated with the degree of progress, to the administrative server 1120 (step S1433).

Thereafter, the AI server 1130 executes learning processing (step S1434). Specifically, for example, the AI server 1130 adds, as the training data, a combination of the fundus image data 103 included in the anonymous diagnostic data received in step S1431 and the degree of progress output in step S1432, to the training data set in the learning DB1131. The AI server 1130 updates the learning model based on the added training data set. Subsequently, the AI server 1130 terminates the processing on the received fundus image and is then brought into a standby state for receiving a next fundus image to be received.

The administrative server 1120 receives the anonymous diagnostic result data when the anonymous diagnostic result data is transmitted thereto from the AI server 1130 (step S1424). Then, the administrative server 1120 restores the patient information 107 based on the anonymous ID included in the anonymous diagnostic result data (step S1425).

For example, the administrative server 1120 reads out the patient information 107 saved in step S1422 and including the patient ID linked to the anonymous ID. Then, the administrative server 1120 generates diagnosis result data including the obtained patient information 107 and the diagnosis results (the degree of progress and the observation 105) (step S1426) and transmits the diagnosis result data to the in-hospital server 1113 (step S1427). Thereafter, the administrative server 1120 saves the diagnosis result data (step S1428) and subsequently terminates its processing.

The in-hospital server 1113 receives the diagnosis result data when the diagnosis result data is transmitted thereto from the administrative server 1120 (step S1414). Next, the terminal 1112 displays, on the display screen 110, the fundus image 103, the degree of progress, the observation 105, and the patient information 107 using the diagnosis result data transmitted from the in-hospital server 1113 and the fundus image data 103 as shown in FIG. 1 or FIG. 2 (step S1415). Then, an end button (not shown) is operated on the user's instruction, thereby terminating the display processing (step S1415).

### <Display Processing Performed by Terminal 1112>

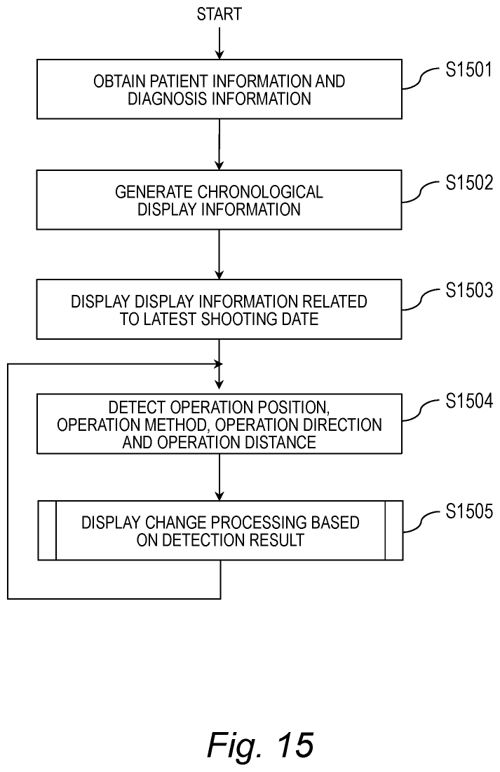

FIG. 15 is a flowchart showing an example of a detailed processing procedure of the display processing (step S1415) performed by the terminal 1112 and shown in FIG. 14. The terminal 1112 obtains patient information 107 and diagnosis information Di1 to Dij of the patient i from the in-hospital server 1113 (step S1501). The terminal 1112 generates chronological display information based on the obtained diagnosis information (step S1502). The chronological display information is a data sequence in which the fundus image data 103 and the observations 105 in the diagnosis information Di1 to Dij are aligned in order of the shooting date Tij. The degree of progress is linked to the corresponding fundus image data 103.

Next, as shown in FIG. 1 or 2, the terminal 1112 displays, on the display screen 110, a fundus image 103, a degree of progress, and an observation 105 at the latest shooting date Tij as display information related to the latest shooting date Tij (step S1503). Thereafter, the terminal 1112 detects an operation position, an operation method, an operation direction and an operation distance L of the user's finger (step S1504). Then, as shown in FIGS. 3 to 10, the terminal 1112 executes the display change processing based on the detection result of step S1504 (step S1505).

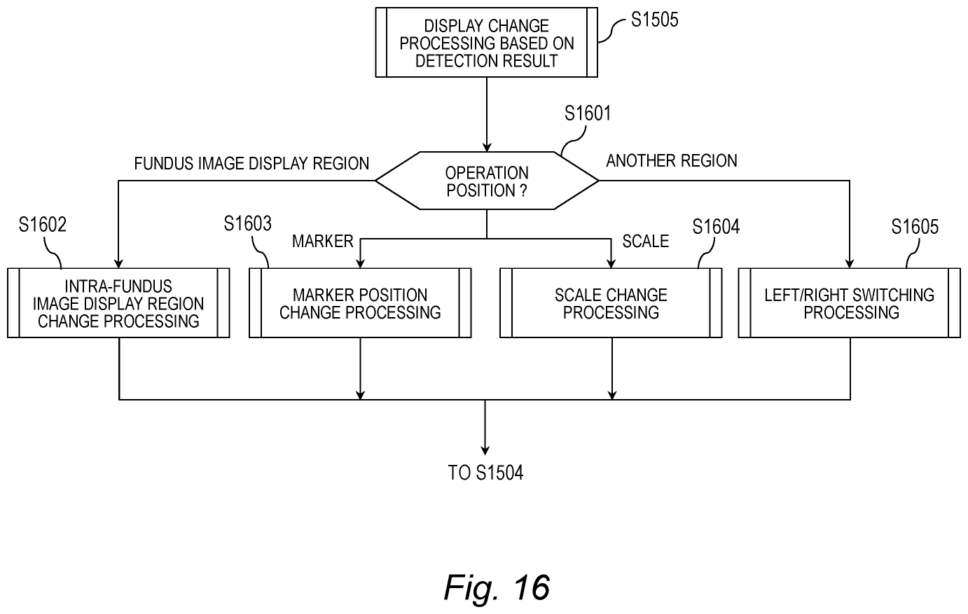

FIG. 16 is a flowchart showing an example of a detailed processing procedure of the display change processing (step S1504) based on the detection result shown in FIG. 15. The terminal 1112 determines whether the operation position of the user's finger is the fundus image display region 104, the marker 102, the scale 101, or another region (step S1601). When the operation position is the fundus image display region 104 (step S1601: fundus image display region), the terminal 1112 executes intra-fundus image display region change processing (step S1602) and returns to step S1504.

When the operation position is the marker 102 (step S1601: marker), the terminal 1112 executes marker position change processing (step S1603) and returns to step S1504. When the operation position is the scale 101 (step S1601: scale 101), the terminal 1112 executes scale change processing (step S1604) and returns to step S1504. When the operation position is another region (step S1601: another region), the terminal 1112 executes left/right switching processing (step S1605) and returns to step S1504.

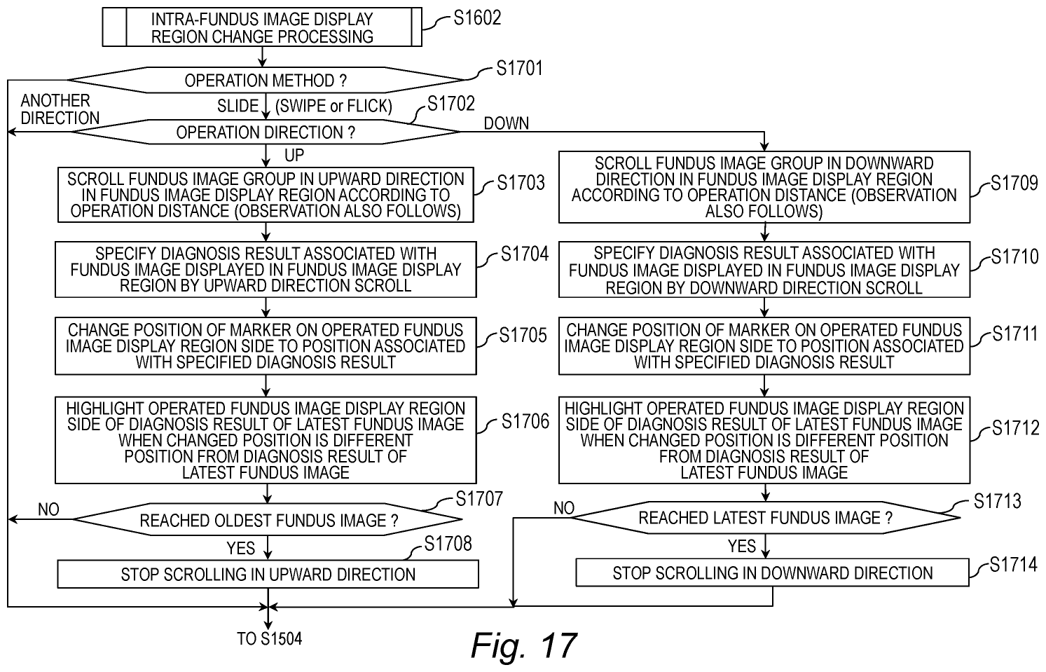

FIG. 17 is a flowchart showing an example of a detailed processing procedure of the intra-fundus image display region change processing (step S1602) shown in FIG. 16. In FIG. 17, when the fundus image group is scrolled in the upward direction D1, a fundus image 103 of an older shooting date Tij than that of the fundus image 103 being displayed appears, whereas when the fundus image group is scrolled in the downward direction D2, a fundus image 103 of a newer shooting date Tij than that of the fundus image being displayed appears. In FIG. 17, the change of the fundus image 103R in the fundus image display region 104R is described as an example, but the same is applied to the fundus image display region 104L.

The terminal 1112 detects an operation method (step S1701). When the operation method is an operation (for example, swiping or flicking) of sliding the finger on the display screen 110 (step S1701: sliding), the processing proceeds to step S1702. When the operation method is another operation method excluding the sliding (step S1701: another operation method), the processing returns to step S1504.

In step S1702, the terminal 1112 detects an operation direction of the user's finger (step S1702). When the operation direction is the upward direction D1 (step S1702: upward), the processing proceeds to step S1703. When the operation direction is the downward direction D2 (step S1702: downward), the processing proceeds to step S1709. When the operation direction is another direction excluding the upward direction D1 and the downward direction D2 (step S1702: another direction), the processing proceeds to step S1504.

When the operation direction is the upward direction D1 (step S1702: upward), the terminal 1112 scrolls through the fundus image group in the upward direction D1 according to the operation distance L within the fundus image display region 104 where the finger is placed (step S1703). An observation group is also scrolled in the upward direction D1 while following the fundas image group.

Then, the terminal 1112 specifies a diagnosis result (the degree of progress and the observation 105) associated with the fundus image which is displayed in the fundus image display region 104 by the scrolling in the upward direction D1 (step S1704). Subsequently, the terminal 1112 changes the position of the marker 102 on the operated fundus image display region 104 side to a position associated with the specified degree of progress (step S1705).

Then, the terminal 1112 may highlight the operated fundus image display region 104 side of the degree of progress corresponding to the latest fundus image 103 if the changed position of the marker 102 is a position different from that of the degree of progress corresponding to the latest fundus image 103 (step S1706). Specifically, the terminal 1112 highlights the marker 120 as shown in FIG. 2. Thus, the user can intuitively identify the degree of progress corresponding to the scrolled fundus image 103.

Subsequently, the terminal 1112 determines whether or not the fundus image displayed in the fundus image display region 104 by the scrolling reaches the oldest fundus image 103 (step S1707). When it does not reach the oldest fundus image 103 (step S1707: No), the processing returns to step S1504. On the other hand, when it reaches the oldest fundus image 103 (step S1707: Yes), the terminal 1112 stops the scrolling in the upward direction D1 (step S1708), and then the processing returns to step S1504.

When the operation direction is the downward direction D2 (step S1702: downward), the terminal 1112 scrolls through the fundus image group in the downward direction D2 according to the operation distance L within the fundus image display region 104 where the finger is placed (step S1709). An observation group is also scrolled in the downward direction D2 while following the fundas image group.

Then, the terminal 1112 specifies a diagnosis result (the degree of progress and the observation 105) associated with the fundus image 103 which is displayed in the fundus image display region 104 by the scrolling in the downward direction D2 (step S1710). Subsequently, the terminal 1112 changes the position of the marker 102 on the operated fundus image display region 104 side to a position associated with the specified degree of progress (step S1711).

Then, the terminal 1112 may highlight the operated fundus image display region 104 side of the degree of progress corresponding to the latest fundus image 103 if the changed position of the marker 102 is a position different from that of the degree of progress corresponding to the latest fundus image 103 (step S1712). Specifically, the terminal 1112 highlights the marker 120 as shown in FIG. 2. Thus, the user can intuitively identify the degree of progress corresponding to the scrolled fundus image 103.

Subsequently, the terminal 1112 determines whether or not the fundus image displayed in the fundus image display region 104 by the scrolling reaches the latest fundus image 103 (step S1713). When it does not reach the latest fundus image 103 (step S1713: No), the processing returns to step S1504. On the other hand, when it reaches the latest fundus image 103 (step S1714: Yes), the terminal 1112 stops the scrolling in the downward direction D2 (step S1714), and then the processing returns to step S1504.

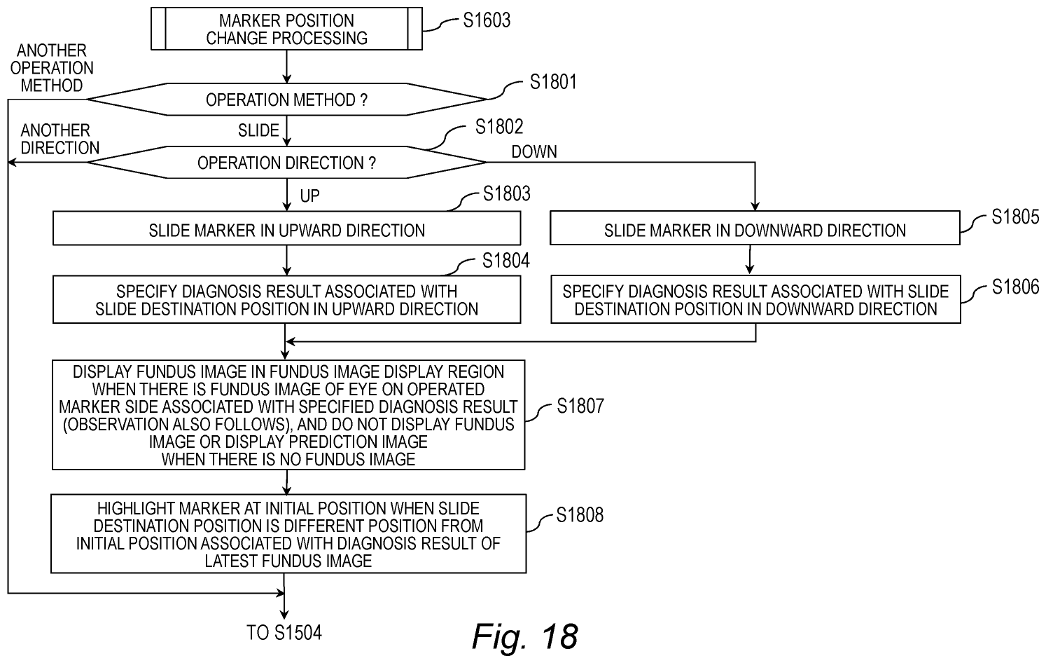

FIG. 18 is a flowchart showing an example of a detailed processing procedure of the marker position change processing (step S1603) shown in FIG. 16. In FIG. 18, when the finger is slid on the marker 102 in the upward direction D1, the marker 102 moves to the position indicating the degree of progress with lower severity, whereas when the finger is slid on the marker 102 in the downward direction D2, the marker 102 moves to the position indicating the degree of progress with higher severity. In FIG. 18, the change of the position of the marker 102L is described as an example, but the same is applied to the marker 102R.

The terminal 1112 detects an operation method (step S1801). When the operation method is an operation (for example, swiping) of sliding the finger on the display screen 110 (step S1801: sliding), the processing proceeds to step S1802. When the operation method is another operation method excluding the sliding (step S1801: another operation method), the processing returns to step S1504.

In step S1802, the terminal 1112 detects an operation direction of the user's finger (step S1802). When the operation direction is the upward direction D1 (step S1802: upward), the processing proceeds to step S1803. When the operation direction is the downward direction D2 (step S1802: downward), the processing proceeds to step S1805. When the operation direction is another direction excluding the upward direction D1 and the downward direction D2 (step S1802: another direction), the processing returns to step S1504.

When the operation direction is the upward direction D1 (step S1802: upward), the terminal 1112 slides the marker 102 where the finger is placed, in the upward direction D1 (step S1803). Subsequently, the terminal 1112 specifies a diagnosis result (the degree of progress and the observation 105) associated with a slide destination position in the upward direction D1 (step S1804), and then the processing proceeds to step S1807.

When the operation direction is the downward direction D2 (step S1702: downward), the terminal 1112 slides the marker 102 where the finger is placed, in the downward direction D2 (step S1805). Subsequently, the terminal 1112 specifies a diagnosis result (the degree of progress and the observation 105) associated with a slide destination position in the downward direction D2 (step S1806), and then the processing proceeds to step S1807.

Then, when there is a fundus image 103 of the eye on the operated marker 102 side associated with the degree of progress specified in step S1804 or S1806, the terminal 1112 displays this fundus image 103 in the fundus image display region 104 (step S1807). The terminal 1112 also displays the observation 105 while following the fundus image. Then, when there is no fundus image 103 of the eye on the operated marker 102 side associated with the degree of progress specified in step S1804 or S1806, the terminal 1112 does not display the fundus image 103 in the fundus image display region 104 or displays a predicted fundus image.

Subsequently, if the slide destination position of the marker 102 is a position different from an initial position associated with the degree of progress corresponding to the latest fundus image 103, the terminal 1112 highlights the marker 102 at the initial position (step S1808), and the processing returns to step S1504. Thus, the user can intuitively identify the initial position of the marker 102 associated with the degree of progress of the fundus image.

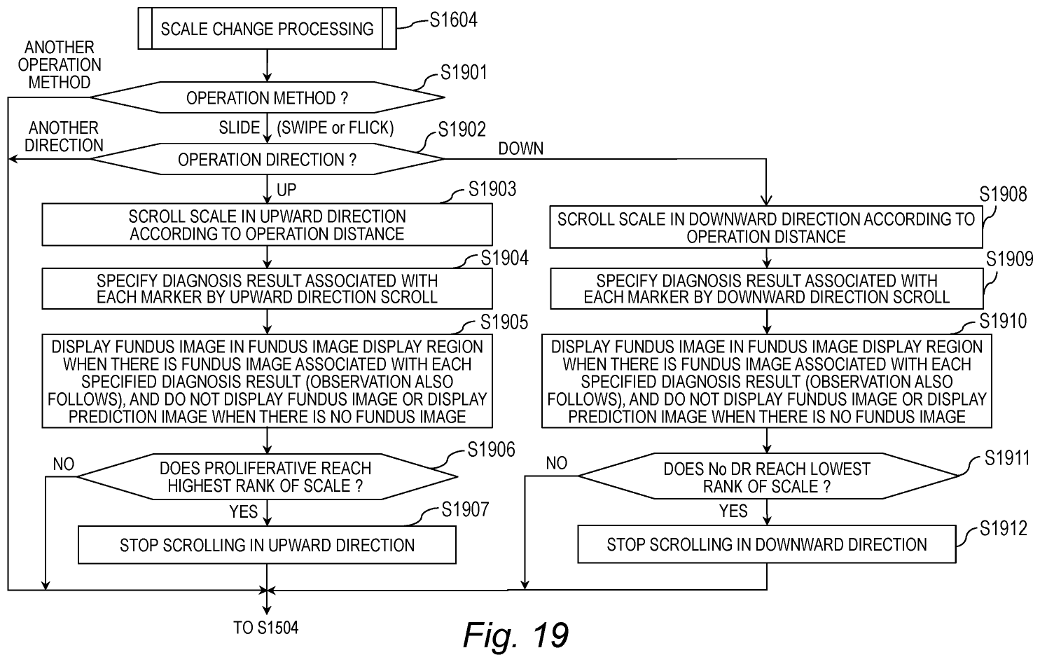

FIG. 19 is a flowchart showing an example of a detailed processing procedure of the change processing of the scale 101 (step S1604) shown in FIG. 16. In FIG. 19, when the finger is slid on the scale 101 in the upward direction D1, the degree of progress is scrolled in the upward direction D1, whereas when the finger is slid on the scale 101 in the downward direction D2, the degree of progress is scrolled in the downward direction D2.

The terminal 1112 detects an operation method (step S1901). When the operation method is an operation (for example, swiping or flicking) of sliding the finger on the scale 101 (step S1901: sliding), the processing proceeds to step S1902. When the operation method is another operation method excluding the sliding (step S1901: another operation method), the processing returns to step S1504.

In step S1902, the terminal 1112 detects an operation direction of the user's finger (step S1902). When the operation direction is the upward direction D1 (step S1902: upward), the processing proceeds to step S1903. When the operation direction is the downward direction D2 (step S1902: downward), the processing proceeds to step S1908. When the operation direction is another direction excluding the upward direction D1 and the downward direction D2 (step S1902: another direction), the processing proceeds to step S1504.

When the operation direction is the upward direction D1 (step S1902: upward), the terminal 1112 slides the plurality of degrees of progress in the upward direction D1 according to the operation distance L (step S1903). Subsequently, the terminal 1112 specifies a diagnosis result (the degree of progress and the observation 105) associated with each marker 102 by the scrolling in the upward direction D1 (step S1904).

Then, when there is a fundus image 103 associated with each degree of progress specified in step S1904, the terminal 1112 displays the fundus image 103 in the fundus image display region 104 (step S1905). The terminal 1112 also displays the observation 105 while following the fundus image. Then, when there is no fundus image 103 associated with each degree of progress specified in step S1904, the terminal 1112 does not display the fundus image in the fundus image display region 104 or displays a predicted fundus image.

Subsequently, the terminal 1112 determines whether or not the degree of progress “Proliferative” reaches the highest rank of the scale 101 (step S1906). When it does not reach the highest rank (step S1906: No), the processing returns to step S1504. On the other hand, when it reaches the highest rank (step S1906: Yes), the terminal 1112 stops the scrolling through the degrees of progress in the upward direction D1 (step S1907), and then the processing returns to step S1504.

When the operation direction is the downward direction D2 (step S1902: downward), the terminal 1112 slides the plurality of degrees of progress in the downward direction D2 according to the operation distance L (step S1908). Subsequently, the terminal 1112 specifies a diagnosis result (the degree of progress and the observation 105) associated with each marker 102 by the scrolling in the downward direction D2 (step S1909).

Then, when there is a fundus image 103 associated with each degree of progress specified in step S1908, the terminal 1112 displays the fundus image 103 in the fundus image display region 104 (step S1910). The terminal 1112 also displays the observation 105 while following the fundus image. Then, when there is no fundus image 103 associated with each degree of progress specified in step S1908, the terminal 1112 does not display the fundus image 103 in the fundus image display region 104 or displays a predicted fundus image 103.

Subsequently, the terminal 1112 determines whether or not the degree of progress “No DR” reaches the lowest rank of the scale 101 (step S1911). When it does not reach the lowest rank (step S1911: No), the processing returns to step S1504. On the other hand, when it reaches the lowest rank (step S1911: Yes), the terminal 1112 stops the scrolling through the degrees of progress in the downward direction D2 (step S1912), and then the processing returns to step S1504.

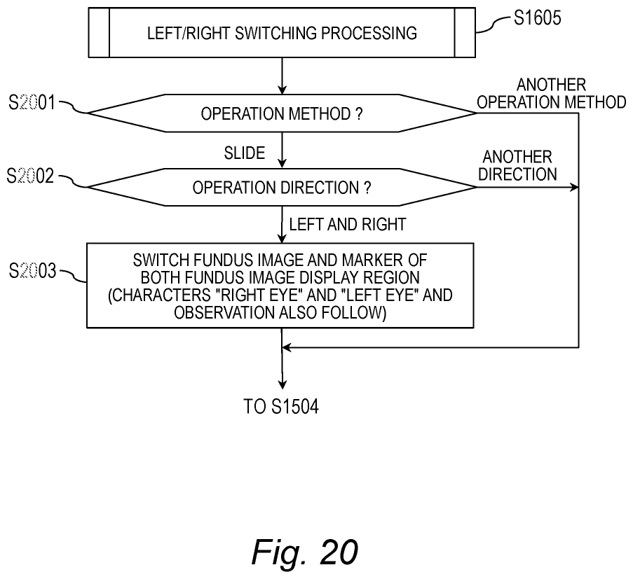

FIG. 20 is a flowchart showing an example of a detailed processing procedure of the left/right switching processing (step S1605) shown in FIG. 16. The terminal 1112 detects an operation method (step S2001). When the operation method is an operation (for example, swiping or flicking) of sliding the finger on the display screen 110 (step S2001: sliding), the processing proceeds to step S2002. When the operation method is another operation method excluding the sliding (step S2001: another operation method), the processing returns to step S1504.

In step S2002, the terminal 1112 detects an operation direction of the user's finger (step S2002). When the operation direction is the left/right direction (step S2002: left/right), the processing proceeds to step S2003. When the operation direction is another direction excluding the left/right direction (step S2002: another direction), the processing proceeds to step S1504.

When the operation direction is the left/right direction (step S2002: left/right), the terminal 1112 switches the fundus image 103 and the marker 102 of the fundus image display region 104 on the left side with those on the right side and vice versa (step S2003). Likewise, the terminal 1112 also performs the same left/right switching processing on the string of characters “right eye” and “left eye” and their observations 105.

### Functional Configuration Example of Information Processing System 1100

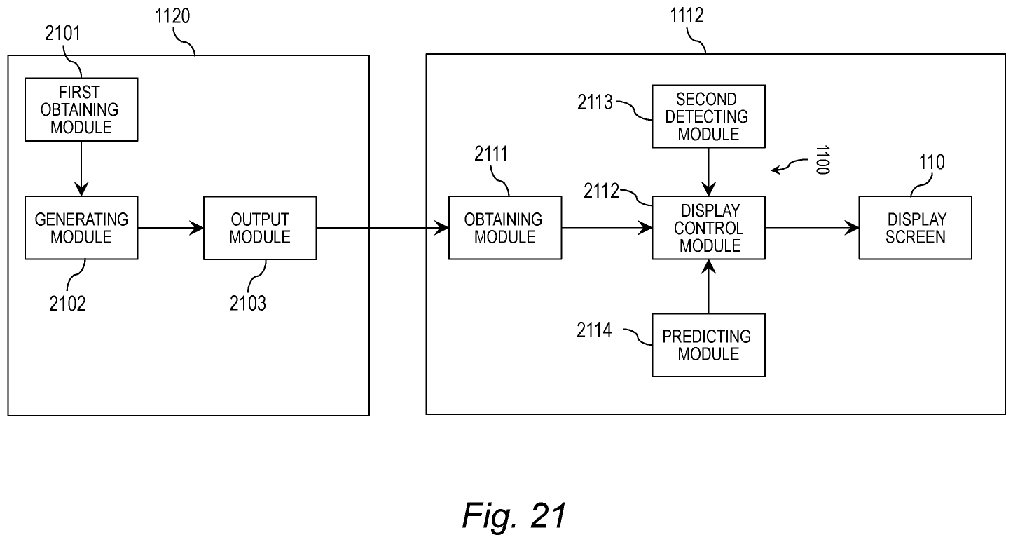

FIG. 21 is a block diagram showing a functional configuration example of the information processing system 1100. In FIG. 21, the functional configurations of the terminal 1112 and the administrative server 1120 are focused and described below, but the in-hospital server 1113 may be described instead of the terminal 1112. The terminal 1112 stores subject eye image data for both eyes of the patient. The administrative server 1120 is communicably connected directly with the terminal 1112 or indirectly with the terminal 1112 via the in-hospital server 1113.

The administrative server 1120 includes a first obtaining module 2101, a generating module 2102, and an output module 2103. Specifically, the first obtaining module 2101, the generating module 2102, and the output module 2103 are realized, for example, by causing a processor 1201 to execute a program stored in the storage device 1202 shown in FIG. 12.

The first obtaining module 2101 obtains anonymous diagnostic result data from the AI server 1130. The generating module 2102 generates the diagnosis result data based on the anonymous diagnostic result data obtained by the first obtaining module 2101. Specifically, the generating module 2102 generates, for example, the diagnosis result display information 100. Specifically, the generating module 2102 specifies the patient ID linked to the anonymous ID included in the anonymous diagnostic result data, and generates the diagnosis result data based on the patient information 107 including the specified patient ID, the diagnosis result included in the anonymous diagnostic result data, and the fundus image data 103 which is the target of the diagnosis. The diagnosis result data is displayed as the diagnosis result display information 100 by the terminal 1112 on the display screen 110. The output module 2103 outputs the diagnosis result data generated by the generating module 2102 directly to the terminal 1112 or indirectly to the terminal 1112 via the in-hospital server 1113.

The terminal 1112 includes the display screen 110, a second obtaining module 2111, a display control module 2112, a detecting module 2113, and a predicting module 2114. Specifically, the second obtaining module 2111, the display control module 2112, the detecting module 2113, and the predicting module 2114 are realized, for example, by causing the processor 1201 to execute a program stored in the storage device 1202 shown in FIG. 12.

The second obtaining module 2111 obtains the diagnosis result data directly from the output module 2103 of the administrative server 1120 or indirectly from the output module 2103 via the in-hospital server 1113. As shown in FIG. 1 or 2, the display control module 2112 displays, on the display screen 110, the patient information 107, the diagnosis result, and the fundus image data 103, which are included in the diagnosis result data.

The detecting module 2113 detects the operation position, operation method, operation direction and operation distance of the user's finger in contact with the display screen 110. As shown in FIGS. 3 to 10, the display control module 2112 executes the display control according to the detection result.

The predicting module 2114 predicts fundus image data 103 associated with the moved marker 102 from the fundus image data 103 associated with the marker 102 before the movement based on the degree of progress specified by the marker 102 before the movement and the degree of progress specified by the moved marker 102. The display control module 2112 displays the fundus image 103 associated with the moved marker 102 as the predicted fundus image in the fundus image display region 104.

Specifically, for example, as shown in FIG. 6, the predicting module 2114 predicts the fundus image data 103L2 associated with the moved marker 102, from the fundus image data 103L1 associated with the marker 102 before the movement when the degree of progress specified by the marker 102 before the movement is “Mild” while the degree of progress specified by the moved marker 102 is “No DR”. In this case, since the severity of the degree of progress is less severe, the predicting module 2114 predicts the fundus image data 103L2 by reducing the size of or eliminating a capillary aneurysm shown in the fundus image 103L1.

As shown in FIG. 7, the predicting module 2114 predicts the fundus image data 103L2 associated with the moved marker 102, from the fundus image data 103L1 associated with the marker 102 before the movement when the degree of progress specified by the marker 102 before the movement is “Mild” while the degree of progress specified by the moved marker 102 is “Moderate”. In this case, since the severity of the degree of progress becomes more severe, the predicting module 2114 predicts the fundus image data 103L2 by increasing the size of a capillary aneurysm shown in the fundus image 103L1 or adding a capillary aneurysm to a further tip portion of a capillary blood vessel.

The predicting module 2114 may determine the amount of change in the size of the capillary aneurysm or the number of capillary aneurysms to be changed in size according to the difference in the degree of progress before and after the movement of the marker 102. For example, in the case of changing from “Mild” to “Moderate”, since a difference in the degree of progress is one level, the predicting module 2114 makes a change corresponding to the one level, in the fundus image data 103L1 associated with the marker 102 before the movement. For example, in the case of changing from “Mild” to “Severe”, since a difference in the degree of progress corresponds to two levels, the predicting module 2114 makes a change corresponding to the two levels, i.e., a larger change than a change corresponding to the one level, in the fundus image data 103L1 associated with the marker 102 before the movement.

Conversely, in the case of changing from “Moderate” to “Mild”, since a difference in the degree of progress is one level, the predicting module 2114 makes a change corresponding to the one level, in the fundus image data 103L1 associated with the marker 102 before the movement. For example, in the case of changing from “Moderate” to “No DR”, since a difference in the degree of progress corresponds to two levels, the predicting module 2114 makes a change corresponding to the two levels, i.e., a larger change than the one level to reduce the size of the capillary aneurysm, in the fundus image data 103L1 corresponding to the marker 102 before the movement.

It is noted that instead of the predicting module 2114, the second obtaining module 2111 transmits a prediction request directly to the AI server 1130 or indirectly to the AI server 1130 via the administrative server 1120 or the in-hospital server 1113. The prediction request includes, for example, the degree of progress specified by the marker 102 before the movement, the degree of progress specified by the moved marker 102, and the fundus image data 103 associated with the marker 102 before the movement. Subsequently, the second obtaining module 2111 may obtain the fundus image data 103 associated with the moved marker 102, which serves as the predicted result, directly from the AI server 1130 or indirectly from the AI server 1130 via the administrative server 1120 or the in-hospital server 1113.

In this case, the AI server 1130 obtains, as a predicted result, fundus image data 103 similar to the subject eye image data associated with the marker 102 before the movement, from a group of the fundus image data 103 matching with the degree of progress specified by the moved marker 102, in the training data set. Further, the AI server 1130 may input the subject eye image data associated with the marker 102 before the movement to the learning model and may then adjust the size of the capillary aneurysm in the subject eye image data associated with the marker 102 before the movement until an output from the learning model becomes the degree of progress specified by the moved marker 102.

In this case, when the degree of progress specified by the marker 102 before and after the movement changes in the direction of increasing the severity, the AI server 1130 increases the size of the capillary aneurysm or adds another capillary aneurysm, whereas when the degree of progress specified by the marker 102 before and after the movement changes in the direction of decreasing the severity, the AI server 1130 decreases the size of the capillary aneurysm or eliminates the capillary aneurysm.

In this way, according to the present embodiment, the common scale 101 for specifying each of the degrees of progress of the fundus images 103 of both eyes is disposed between the fundus images 103 on the display screen 110, thereby making it possible to improve the visibility of the diagnosis information. Furthermore, by disposing such a scale 101, the space of the display screen 110 can be saved.

In addition, by manipulating the fundus image 103 through the user interface, the display can be easily changed from the fundus image 103 being displayed to another fundus image 103 before or after the date of shooting. Since the position of the marker 102 and the observation 105 are changed in conjunction with the display change of the fundus image 103, the degree of progress corresponding to the fundus image 103 and the observation 105 after the display change can be confirmed easily even after the display change of the fundus image 103.

Further, in conjunction with the position change of the marker 102 through the user interface, the display can be changed from the fundus image 103 associated with the degree of progress before the position change of the marker 102 to the fundus image 103 associated with the degree of progress after the position change of the marker 102. This allows the user to easily confirm how severe the degree of progress of the symptom of the fundus is. When there is no fundus image 103 after the position change of the marker 102, this fundus image 103 is predicted, thus also allowing the user to easily confirm how severe the degree of progress of the symptom of the fundus is.

By scrolling through the degrees of progress, the degrees of progress of both eyes are changed while maintaining a difference between these degrees of progress, and concurrently in conjunction with the change of the degrees of progress, the display is changed from the fundus images 103 of both eyes before the change of the degrees of progress to the fundus image 103 of both eyes after the change of the degrees of progress. This allows the user to easily confirm how severe the degree of progress of the symptom of the fundus is. How both eyes change with the progress of the disease can be easily confirmed in a single operation.

When there is no fundus image 103 after the position change of the marker 102, this fundus image 103 is predicted, thus also allowing the user to easily confirm how severe the degree of progress of the symptom of the fundus is. Since the observations 105 of both eyes are also changed in conjunction with the display change of the fundus images 103 of both eyes, the observations 105 of both eyes after the display change can be easily confirmed even after the display change of the fundus images 103 of both eyes.

The present invention is not limited to the above-mentioned contents, and these contents may be optionally used in combination. Other embodiments that are considered within the scope of the technical idea of the present invention are also included in the scope of the present invention.

### EXPLANATION OF SYMBOLS

100 diagnosis result display information, 101 scale, 102, 120 marker, 103 fundus image (fundus image data), 104 fundus image display region, 105 observation, 106 observation display region, 107 patient information, 110 display screen, 1100 information processing system, 1101 in-hospital system, 1111 ophthalmic device, 1112 terminal, 1113 in-hospital server, 1114 patient information DB, 1120 administrative server, 1130 AI server, 1131 learning DB, 1200 computer, 1201 processor, 1202 storage device, 2101 first obtaining module, 2102 generating module, 2103 output module, 2111 second obtaining module, 2112 display control module, 2113 detecting module, 2114 predicting module, Dij diagnosis information

1.-39. (canceled)

40. An information processing apparatus comprising:
a processor; and
a machine-readable medium encoded with instructions executed by the processor, the instruction causing the processor to perform operations comprising:
obtaining image analysis data of an eye which includes the image data of the eye, a symptom of eye disease detected on the image data of the eye, and severity of the symptom of the eye disease; and
generating a user interface which includes the image of the eye, the symptom of the eye disease, the severity of the symptom of the eye disease, and a scale used as a measure of the symptom severity of the eye disease.

41. The information processing apparatus according to claim 40, wherein the image data includes image data of right and left eyes of a person, a symptom of the eye disease detected on the image data of the right and left eyes, respectively, and severity of the symptom of the eye disease on the right and left eyes, respectively,
wherein the instruction causes the processor to generate the user interface which includes the images of the right and left eyes, the symptom of the eye disease for the right and left eyes, the severity of the symptom of the eye disease for the right and left eyes, and the scale being arranged between the right eye image and the left eye image.

42. The information processing apparatus according to claim 40, wherein the instruction causes the processor to perform operations comprising:
obtaining present image analysis data of the eye which includes a set of present image data of the eye, a present symptom of eye disease detected on the present image of the eye, and present severity of the symptom of the eye disease;
obtaining past image analysis data of the eye which includes a set of past image data, a past symptom of the eye disease detected on the past image of the eye, and past severity of the symptom of the eye disease;
displaying the user interface which includes the present image, the present symptom of the eye disease, and the severity of the symptom of the eye disease;
detecting a user operation on the user interface; and
upon the user operation, enabling to display on the user interface the past image of the eye, the past symptom of the eye disease, and the past severity of the symptom of the eye disease.

43. The information processing apparatus according to claim 42, wherein the past image analysis data includes plural sets of past image data of the eye, past symptom of the eye disease detected on the past image data and past severity of the symptom of the eye disease, each of the plural sets of past image data, past symptom of the eye disease and past severity of the symptom of the eye disease being obtained at different times,
wherein the instruction causes the processor to perform operations, further comprising, in response to the user operation, selecting a past image of the eye and/or past severity of the symptom to be displayed from among the plural sets of past image data and past severity of the symptom.

44. The information processing apparatus according to claim 43, wherein the instruction causes the processor to perform operations comprising:
in the detecting the user operation, enabling to select severity of the symptom of the eye disease on the scale, and
in response to the user operation, selecting a past image of the eye to be displayed from among the plural sets of past image analysis data, the severity of the symptom associated with the selected past image data corresponding to the severity of the symptom selected on the scale.

45. The information processing apparatus according to claim 40, wherein the instruction causes the processor to perform operations comprising:
detecting a user operation on the scale of the user interface;
upon the detecting the user operation, enabling to select severity of the symptom of the eye disease on the scale; and
obtaining a predictable image data of the eye, severity of the symptoms on the predictable image data corresponding to the severity of the symptom selected on the scale.

46. The information processing apparatus according to claim 45, wherein the predictable image data is associated with increased symptom of the eye disease and generated based upon the image data of the eye and the symptom of the eye disease detected on the image data.

47. The information processing apparatus according to claim 45, wherein the predictable image data is associated with no symptom of the eye disease and generated based upon the image data of the eye and the symptom of the eye disease detected on the image data.

48. An information processing apparatus comprising:
a processor; and
a machine-readable medium encoded with instructions executed by the processor, the instruction causing the processor to perform operations comprising:
obtaining image analysis data of an eye which includes the image data of the eye, a symptom of eye disease detected on the image data of the eye;
determining severity of the symptom of the eye disease based upon the image analysis data; and
generating a user interface which includes the image of the eye, the symptom of the eye disease, the severity of the symptom of the eye disease, and a scale used as a measure of the symptom severity of the eye disease.

49. The information processing apparatus according to claim 48, wherein the image data includes image data of right and left eyes of a person, and symptom of the eye disease detected on the image data of the right and left eyes, respectively,
wherein the instruction causes the processor to perform operations comprising:
in the determining severity, determining severity of the symptom of the eye disease on the right and left eyes, respectively; and
in the generating a user interface, generating the user interface which includes the images of the right and left eyes, the symptom of the eye disease for the right and left eyes, the severity of the symptom of the eye disease for the right and left eyes, and the scale being arranged between the right eye image and the left eye image.

50. The information processing apparatus according to claim 48, wherein the instruction causes the processor to perform operations comprising:
obtaining present image analysis data of the eye which includes a set of present image data of the eye, a present symptom of eye disease detected on the present image of the eye, and present severity of the symptom of the eye disease;
obtaining past image analysis data of the eye which includes a set of past image data, a past symptom of the eye disease detected on the past image of the eye, and past severity of the symptom of the eye disease;
displaying the user interface which includes the set of the present image, the present symptom of the eye disease, and the severity of the symptom of the eye disease;
detecting a user operation on the user interface; and
upon the user operation, enabling to display on the user interface the set of the past image of the eye, the past symptom of the eye disease, and the past severity of the symptom of the eye disease.

51. The information processing apparatus according to claim 50, wherein the past image analysis data includes plural sets of past image data of the eye, past symptom of the eye disease detected on the past image data and past severity of the symptom of the eye disease, each of the plural sets of past image data, past symptom of the eye disease and past severity of the symptom of the eye disease being obtained at different times,
wherein the instruction causes the processor to perform operations, further comprising, in response to the user operation, selecting a past image of the eye and/or past severity of the symptom to be displayed from among the plural sets of past image data, past symptom of the eye disease and past severity of the symptom.

52. The information processing apparatus according to claim 51, wherein the instruction causes the processor to perform operations comprising:
in the detecting the user operation, enabling to select severity of the symptom of the eye disease on the scale, and
in response to the user operation, selecting past image data of the eye to be displayed from among the plural sets of past image analysis data, severity of the symptom associated with the selected past image data corresponding to the severity of the symptom selected on the scale.

53. The information processing apparatus according to claim 48, wherein the instruction causes the processor to perform operations comprising:
detecting a user operation on the scale of the user interface;
upon the detecting the user operation, enabling to select severity of the symptom of the eye disease on the scale; and
generating a predictable image data based upon the image analysis data of the eye, severity of the symptom on the predictable image data corresponding to the severity of the symptom selected on the scale.

54. The information processing apparatus according to claim 53, wherein the predictable image is associated with increased symptom of the eye disease and generated based upon the image data of the eye and the symptom of the eye disease detected on the image data.

55. The information processing apparatus according to claim 53, wherein the predictable image is associated with no symptom of the eye and generated based upon the image data of the eye and the symptom of the eye disease detected on the image data.

56. An information processing system comprising:
a first information processing apparatus configured to:
obtain image data of an eye;
perform an image analysis on the image data of the eye to detect a symptom of eye disease; and
generate image analysis data of the eye including the image data of the eye and the symptom of eye disease detected on the image data of the eye;
a second information processing apparatus configured to:
obtain the image analysis data from the first information apparatus; and
generate a user interface including the image of the eye and the symptom of the eye disease.

57. The information processing system according to claim 55, wherein the first information processing apparatus or the second information processing apparatus is configured to determine severity of the symptom of the eye disease based upon the image analysis data, the severity of the symptom of the eye disease being included in the image analysis data,
wherein the second information processing apparatus is configured to generate the user interface further including the severity of the symptom of the eye disease and a scale used as a measure of the symptom severity of the eye disease.

58. The information processing system according to claim 57, wherein the second information processing device is configured to:
detect a user operation on the scale of the user interface;
upon the detecting the user operation, enable to select severity of the symptom of the eye disease on the scale; and
obtain a predictable image data being associated with severity of the symptom of the eye disease which corresponds to the severity of the symptom selected on the scale, the predictable image data being generated by the first information processing device or the second information processing device based upon the image analysis data.

59. The information processing system according to claim 56, wherein the second information processing device is configured to
obtain from the first information apparatus present image analysis data of the eye which includes a set of present image data of the eye and a present symptom of eye disease detected on the present image of the eye;
obtain plural sets of past image data of the eye and past symptom of the eye disease detected on the past image data, each of the plural sets of past image data and past symptom of the eye disease being obtained at different times, the plural sets of past image analysis data being stored on a storage device of the second information processing device or transmitted from the first information processing device;
display the user interface which includes the present image of the eye and the present symptom of eye disease detected on the present image of the eye;
detect a user operation on the user interface; and
in response to the user operation, select a past image of the eye to be displayed from among the plural sets of past image data.
